(19) United States
(12) Patent Application Publication
(10) Pub. No.: US 2023/0103352 A1
(43) Pub. Date: Apr. 6, 2023

(54) METHODS OF USE OF SOLUBLE CD24 FOR TREATING SARS-COV-2 INFECTION

(71) Applicant: Oncoimmune, Inc., Rahway, NJ (US)

(72) Inventors: Xianfeng Fang, Guangdong (CN); Yang Liu, Potomac, MD (US); Pan Zheng, Potomac, MD (US); Martin Devenport, Gaithersburg, MD (US); Dongling Li, Jiangsu (CN); Libing Mu, Guangdong (CN)

(73) Assignee: Oncoimmune, Inc., Rahway, NJ (US)

(21) Appl. No.: 17/798,481

(22) PCT Filed: Feb. 10, 2021
(86) PCT No.: PCT/CN2021/076544

§ 371 Date: Aug. 9, 2022

Related U.S. Application Data
(60) Provisional application No. 63/083,012, filed on Sep. 24, 2020.

(30) Foreign Application Priority Data
Feb. 10, 2020 (CN) 202010086132.6

(51) Int. Cl. A61K 38/17; A61P 31/14

(52) U.S. Cl. CPC A61K 38/177; A61P 31/14

(57) ABSTRACT

Provided is the use of a CD24 protein for treating a severe acute respiratory syndrome coronavirus 2 (SARS-CoV-2) infection.

### STATEMENT REGARDING FEDERALLY SPONSORED RESEARCH OR DEVELOPMENT

This invention was made, in part, with Government support under NIH/NCI Grant Number 3 R44 CA246991-02S1. The Government has certain rights in this invention

### FIELD OF THE INVENTION

The present invention relates to compositions and methods for treating a severe acute respiratory syndrome coronavirus 2 (SARS-CoV-2) infection.

### BACKGROUND OF THE INVENTION

SARS-CoV-2 is a major health concern affecting millions of people across the globe. An intriguing possibility is that the effects of SARS-CoV-2 may result in release of danger-associated molecular patterns (also referred to as damage-associated molecular patterns or DAMPS) that cause a self-propagating inflammatory response with lasting lung damage. Accordingly, it is of interest to treat coronaviruses, including SARS-CoV, SARS-CoV-2 and MERS infections by targeting DAMP-induced inflammation.

### SUMMARY OF THE INVENTION

This invention relates to a method of treating a coronavirus infection, including SARS-CoV, SARS-CoV-2, or MERS in a subject in need thereof, comprising administering to the subject an effective amount of a CD24 protein as described herein. The invention also relates to the use of the CD24 protein in the manufacture of a medicament for treating a coronavirus infection in a subject in need thereof. An embodiment of the invention is realized when the coronavirus is SARS-CoV. Another embodiment of this aspect of the invention is realized when the coronavirus is SARS-CoV-2. A subembodiment of this aspect of the invention is realized when the SARS-CoV-2 infection is coronavirus disease 2019 (COVID-19). Another embodiment of this aspect of the invention is realized when the coronavirus is MERS. An embodiment of the invention is realized when the coronavirus is a human coronavirus. Another embodiment of this aspect of the invention is when the coronavirus is human SARS-CoV. Another embodiment of this aspect of the invention is realized when the coronavirus is human SARS-CoV-2. Another embodiment of this aspect of the invention is realized when the coronavirus is human MERS.

In one embodiment of the disclosed method, the CD24 protein comprises a mature CD24 polypeptide or variant thereof. In another embodiment the CD24 protein comprises a mature human CD24 polypeptide or variant thereof. In another embodiment the mature human CD24 polypeptide is a polypeptide of SEQ ID NO. 1 or SEQ ID NO. 2. In another embodiment the mature human CD24 polypeptide is a polypeptide of SEQ ID NO. 1. In another embodiment the mature human CD24 polypeptide is a polypeptide of SEQ ID NO. 1, wherein the C-terminal amino acid is valine. In another embodiment the mature human CD24 polypeptide is a polypeptide of SEQ ID NO. 1, wherein the C-terminal amino acid is alanine. In another embodiment the mature human CD24 polypeptide is a polypeptide of SEQ ID NO. 2.

In another embodiment of the disclosed method, the CD24 protein comprises a protein tag. An aspect of this embodiment is realized when the protein tag is fused to the N-terminus or C-terminus of the mature CD24 polypeptide or variant thereof. Another aspect of this embodiment is realized when the protein tag comprises a portion of a mammalian immunoglobulin (Ig) protein. Another aspect of this embodiment is realized when the portion of the mammalian Ig protein is a Fc region. In another embodiment of the disclosed method, the CD24 protein comprises a protein tag fused to the N-terminus or C-terminus of a mature human CD24 polypeptide or variant thereof. Another aspect of this embodiment is realized when the protein tag comprises a portion of a human immunoglobulin (Ig) protein. Another aspect of this embodiment is realized when the portion of the human Ig protein is a Fc region. Another aspect of this embodiment is realized when the Fc region comprises a hinge region and CH2 and CH3 domains of a human Ig protein selected from the group consisting of IgG1, IgG2, IgG3, IgG4, and IgA. Another aspect of this embodiment is realized when the Fc region comprises a hinge region and CH2, CH3 and CH4 domains of IgM. Still another aspect of this embodiment is realized when the amino acid sequence of the CD24 protein comprises a sequence set forth in SEQ ID NO: 6, 11, or 12. Another aspect of this embodiment is realized when the amino acid sequence of the CD24 protein comprises a sequence set forth in SEQ ID NO: 6. Another aspect of this embodiment is realized when the amino acid sequence of the CD24 protein comprises a sequence set forth in SEQ ID NO: 11. Another aspect of this embodiment is realized when the amino acid sequence of the CD24 protein comprises a sequence set forth in SEQ ID NO: 12. A further aspect of this embodiment is realized when the amino acid sequence of the CD24 protein consists of a sequence set forth in SEQ ID NO: 6, 11, or 12. Another aspect of this embodiment is realized when the amino acid sequence of the CD24 protein consists of a sequence set forth in SEQ ID NO: 6. Another aspect of this embodiment is realized when the amino acid sequence of the CD24 protein consists of a sequence set forth in SEQ ID NO: 11. Another aspect of this embodiment is realized when the amino acid sequence of the CD24 protein consists a sequence set forth in SEQ ID NO: 12. Another embodiment of this aspect of the invention is realized when the CD24 protein is CD24Fc.

This invention also relates to use of a CD24 protein as disclosed herein in the manufacture of a medicament for treating a SARS-CoV-2 infection.

As illustrated herein, the subject may have or be diagnosed with coronavirus disease, including coronavirus disease 2019 (COVID-19). In one embodiment of the present invention, the dose of the CD24 protein is 480 mg. In another embodiment of the invention, the CD24 protein is administered intravenously, over a period of 30 minutes to 8 hours. In one class of this embodiment, the CD24 protein is administered intravenously over a period of 30, 40, 50, 60, 70, 80, 90, 100, 110, or 120 minutes, or up to 3, 4, 5, 6, 7 or 8 hours. In another class of this embodiment, the CD24 protein is administered intravenously over a period of about 60 minutes. In a further embodiment of the present invention, the CD24 protein is diluted in an appropriate vehicle. In one aspect of this embodiment, the vehicle is phosphate-buffered saline (PBS) normal saline. In a class of this embodiment, the dose of the CD24 protein is diluted to in an appropriate vehicle, such as PBS. In yet another class, the dose of the CD24 protein is diluted to 100 mL, in an appropriate vehicle, such as PBS. In certain embodiments of the present invention, the CD24 protein is administered with a second treatment, such as treatments that are the standard of care treatment for coronavirus infection and/or disease, including SARS-CoV-2 infection and/or COVID-19. The second treatment may be one or more of oxygen therapy, mechanical ventilation, extracorporeal membrane oxygenation, non-invasive ventilation, a high flow oxygen device, remdesivir, a corticosteroid, and an immune modulator. In one example, the subject is also treated with remedesivir or a corticosteroid.

Administering the CD24 protein as part of the disclosed method may result in one or more of: a reduced risk of death; a reduced duration of treatment with mechanical ventilation, a reduced duration of treatment with extracorporeal membrane oxygenation, a reduced duration of treatment with non-invasive ventilation, a reduced duration of treatment with pressors, a reduced duration of treatment with a high flow oxygen device, a decreased rate of disease progression, an increased time to clinical relapse, a reduced duration of supplemental oxygen, a reduced time of hospital stay, a reduced absolute lymphocyte count, reduced levels of one or more markers of inflammation, and reduced levels of D-dimer concentration; as compared to a population of patients with a SARS-CoV-2 infection (or COVID-19) who were treated with a placebo in combination with standard of care. In one example, administering the CD24 protein may result in reduced time to clinical improvement, as measured by the time in days required from administering the CD24 protein to the improvement of clinical status from “scale 3 or 4” to “scale 5 or higher” based on a National Institute of Allergy and Infectious Diseases (MAID) 8-point ordinal scale within 28 days from administering the CD24 protein. The reduced time may be in comparison to a population of patients with a SARS-CoV-2 infection (or COVID-19) who were treated with a placebo in combination with standard of care.

In one embodiment of the present invention, the CD24 protein administered as part of the disclosed method is soluble. In another embodiment of the disclosed method, the CD24 protein is glycosylated.

In one embodiment of the present invention, the CD24 protein administered by the method described herein is produced using a eukaryotic expression system. In one aspect of this embodiment, the expression system comprises a vector contained in a Chinese Hamster Ovary cell line or a replication-defective retroviral vector. In a class of this embodiment, the replication-defective retroviral vector is stably integrated into the genome of the eukaryotic cell.

### BRIEF DESCRIPTION OF THE DRAWINGS

FIGS. 1A-C. FIG. 1A shows the amino acid composition of the full-length version of a CD24 fusion protein (CD24Fc) with a signal peptide (also referred to herein as CD24Ig) (SEQ ID NO: 5). The underlined 26 amino acids (SEQ ID NO: 4) represent the signal peptide, which is cleaved off during secretion from a cell expressing the protein and thus missing from the processed version of the CD24 protein as represented by SEQ ID NO: 6. The bold portion of the sequence (SEQ ID NO: 2) is the extracellular domain of the processed CD24 protein, The last (C-terminal) amino acid (A or V) that is ordinarily present in a mature CD24 protein has been deleted from the fusion protein construct to reduce immunogenicity. The non-underlined, non-bold letters are the sequence of IgG1 Fc, including the hinge region and CH2 and CH3 domains (SEQ ID NO: 7). FIG. 1B shows the sequence of CD24^{v}Fc (SEQ ID NO: 8), in which the mature human CD24 protein (bold) is the valine polymorphic variant of SEQ ID NO: 1. FIG. 1C shows the sequence of CD24^{A}Fc (SEQ ID NO: 9), in which the mature human CD24 protein (bold) is the alanine polymorphic variant of SEQ ID NO: 1. The various parts of the fusion protein in FIGS. 1B and 1C are marked as in FIG. 1A and the variant valine/alanine amino acid is double underlined.

FIG. 2 shows amino acid sequence variations between mature CD24 polypeptides from mouse (SEQ ID NO: 3) and human (SEQ ID NOs: 1 and 2). The potential O-glycosylation sites are bolded, and the N-glycosylation sites are underlined.

FIGS. 3A-C. WinNonlin compartmental modeling analysis of pharmacokinetics of CD24IgG1 (CD24Fc). The opened circles represent the average of 3 mice, and the line is the predicted pharmacokinetic curve. FIG. 3A. intravenous (i.v.) injection of 1 mg CD24IgG1 (CD24Fc). FIG. 3B. subcutaneous (s.c.) injection of 1 mg CD24IgG1 (CD24Fc). FIG. 3C. Comparison of the total amounts of antibody in the blood as measured by areas under curve (AUC), half-life and maximal blood concentration. Note that overall, the AUC and Cmax of the s.c. injection are about 80% of i.v. injection, although the difference is not statistically significant.

FIGS. 4A-B. CD24-Siglec G (10) interaction discriminates between pathogen associated molecular pattern (PAMP) and danger associated molecular pattern (DAMP). FIG. 4A. Host response to PAMP was unaffected by CD24-Siglec G(10) interaction. FIG. 4B. CD24-Siglec G (10) interaction represses host response to DAMP, possibly through the Siglec G/10-associated SHP-1.

FIGS. 5A-C. CD24Fc binds to Siglec 10 and HMGB1 and activates Siglec G, the mouse homologue of human Siglec 10. FIG. 5A. Affinity measurement of the CD24Fc-Siglec 10 interaction. FIG. 5B. CD24Fc specifically interacts with HMGB-1 in a cation-dependent manner. CD24Fc was incubated with HMGB1 in 0.1 mM of CaCl₂ and MgCl₂, in the presence or absence of the cation chelator EDTA. CD24Fc is pulled down with protein G-beads, and the amounts of HMGB1, CD24Fc or control Fc is determined by Western blot. FIG. 5C. CD24Fc activates mouse Siglec G by inducing Tyrosine phosphorylation (middle panel) and association with SHP-1 (upper panel). The amounts of Siglec G are shown in the lower panel. CD24^{-/-} spleen cells were stimulated with 1 μg/ml of CD24Fc, control Fc or vehicle (phosphate-buffered saline, PBS) control for 30 minutes. Siglec G was then immunoprecipitated and probed with anti-phospho-tyrosine or anti-SHP-1.

FIGS. 6A-B. CD24Fc inhibits production of TNF-α (FIG. 6A) and IFN-γ (FIG. 6B) by anti-CD3 activated human T cells. The human peripheral blood mononuclear lymphocytes (PBML) were stimulated with anti-CD3 for 4 days in the presence or absence of CD24Fc and the amounts of IFN-γ and TNF-α released in the supernatant of cell culture were measured by ELISA. Data shown are means of triplicate. Error bar, SEM.

FIGS. 7A-B. CD24 inhibits inflammatory cytokine production by human macrophages. FIG. 7A. ShRNA silencing of CD24 leads to spontaneous production of TNF-α, IL-1β, and IL-6. THP1 cells were transduced with lentiviral vectors encoding either scrambled or two independent CD24 shRNA molecules. The transduced cells were differentiated into macrophages by culturing for 4 days with phorbol 12-myristate 13-acetate (PMA) (15 ng/ml). After washing away PMA and non-adherent cells, the cells were cultured for another 24 hours for measurement of inflammatory cytokines, by cytokine beads array. FIG. 7B. As in FIG. 7A, except that the given concentration of CD24Fc or control IgG Fc was added to macrophages in the last 24 hours. Data shown in FIG. 7A are means and S.D. from three independent experiments, while those in FIG. 7B are representative of at least 3 independent experiments.

FIG. 8. Diagram of study design.

FIG. 9. Kaplan-Meier (K-M) Estimates of Cumulative Time to Clinical Improvement.

FIG. 10. Kaplan-Meier Estimates of Cumulative Time to Clinical Improvement in patients with a baseline of NIAID score of 4 (hospitalized, requires supplemental oxygen support).

FIG. 11. Kaplan-Meier Estimates of Cumulative Time to Clinical Improvement in patients with a baseline of NIAID score of 3 (hospitalized, requires non-invasive mechanical ventilation or high flow oxygen devices).

FIG. 12. Kaplan-Meier Estimates of Cumulative Time to Clinical Improvement in Male patients.

FIG. 13. Kaplan-Meier Estimates of Cumulative Time to Clinical Improvement in Female patients.

FIG. 14. Kaplan-Meier Estimates of Cumulative Time to Clinical Improvement in White patients.

FIG. 15. Kaplan-Meier Estimates of Cumulative Time to Clinical Improvement in Black or African American Patients.

FIG. 16. Kaplan-Meier Estimates of Cumulative Time to Clinical Improvement in Other Races Patients.

FIG. 17. Kaplan-Meier Estimates of Cumulative Time to Clinical Improvement in Hispanic or Latino Patients.

FIG. 18. Kaplan-Meier Estimates of Cumulative Time to Clinical Improvement in Patients Not Hispanic or Latino.

FIG. 19. Kaplan-Meier Estimates of Cumulative Time to Clinical Improvement in Patients with Remdesivir as part of standard of care.

FIG. 20. Kaplan-Meier Estimates of Cumulative Time to Clinical Improvement in

Patients with corticosteroid as part of standard of care.

FIG. 21. Kaplan-Meier Estimates of Cumulative Time to Clinical Improvement, Using Baseline NIAID Score as Covariate.

FIG. 22. Kaplan-Meier Estimates of Cumulative Time to Clinical Improvement, Using Gender as Covariate.

FIG. 23. Kaplan-Meier Estimates of Cumulative Time to Clinical Improvement, Using Race as Covariate.

FIG. 24. Kaplan-Meier Estimates of Cumulative Time to Clinical Improvement, Using Ethnicity as Covariate.

FIG. 25. Death/RF on Day 29 by treatment assignment.

FIG. 26. Time to Disease Progression in CD24Fc and Placebo arms.

FIG. 27. Kaplan-Meier Estimate of mortality in placebo and CD24Fc arms.

FIG. 28. Mortality rate at Day 15.

FIG. 29. Mortality rate at Day 29.

### DETAILED DESCRIPTION

The inventors have discovered that a soluble form of CD24 protein is highly effective for treating a SARS-CoV-2 infection and/or COVID-19. The inventors have determined from a clinical study that patients infected with SARS-CoV-2 and/or COVID-19 who received a CD24 protein described herein had a 60% better chance to achieve clinical recovery than those who received placebo. An aspect of this invention is realized when the CD24 protein is a mature human CD24 polypeptide (SEQ ID NO: 1), or a variant of the mature human CD24 polypeptide wherein the last (C-terminal) amino acid (A or V) of the mature CD24 polypeptide has been deleted (SEQ ID NO: 2). Another aspect of the invention is realized when said mature human CD24 polypeptide or variant thereof is fused to an IgG1 Fc domain. The median time to recovery was 6 days for patients treated with the CD24 protein compared with 10 days in the placebo group. In addition, the risk of death or respiratory failure was reduced by more than 50%. Thus, this invention also relates to a method for reducing the risk of death or respiratory failure in a subject infected with coronavirus disease comprising administering to the subject a CD24 protein as described herein. An embodiment of this aspect of the invention includes a method for the treatment of coronavirus disease COVID-19 in hospitalized patients with confirmed SARS-CoV-2 viral infection and receiving or requiring supplemental oxygen or non-invasive respiratory support comprising administering to the subject a CD24 protein as described herein. An embodiment of this aspect of the invention is a method for the treatment of coronavirus disease COVID-19 in hospitalized patients with confirmed SARS-CoV-2 viral infection and receiving or requiring supplemental invasive respiratory support comprising administering to the subject a CD24 protein as described herein. Since the clinical trial did not exclude other experimental therapeutics, many participants also received remdesivir and/or corticosteroids, including dexamethasone. Among them, those subjects who were treated with the CD24 protein and remdesivir achieved clinical recovery 7 days earlier than those subjects who received remdesivir and placebo (median time to recovery 6 days vs 13 days). Those subjects who were treated with the CD24 protein and corticosteroids achieved clinical recovery 10 days earlier than those subjects who received corticosteroids and placebo (median time to recovery 5 days vs 15 days).

Without being bound by theory, CD24 may influence lung inflammation, lymphopenia (by preventing deletion and functional exhaustion of T cells), cytokine release syndrome (by blocking production of multiple inflammatory cytokines), and/or blood coagulation (by suppressing the expression of multiple genes in the coagulation pathway). The effect of CD24 may be mediated through DAMPs. Pattern recognition is involved in inflammatory response triggered by both pathogen-associated and tissue damage-associated molecular patterns, respectively called PAMPs and DAMPs. Recent studies have demonstrated that an exacerbated host response to DAMPs may play a part in the pathogenesis of inflammatory and autoimmune disease (Chen, G. Y., et al., CD24 and Siglec-10 selectively repress tissue damage induced immune responses, Science, Vol. 323, pp. 1722-1725 (2009); Liu, Y., et al., CD24-Siglec G/10 discriminates danger-from pathogen-associated molecular patterns, Trends Immunol, Vol. 30, pp. 557-561 (2009); Fang, X., et al., CD24: from A to Z. Cell Mol Immunol, Vol. 7, pp. 100-103 (2010)). DAMPs were found to promote the production of inflammatory cytokines and autoimmune diseases in animal models, and inhibitors of DAMPs such as HMGB1 and HSP90 were consequently found to ameliorate rheumatoid arthritis (RA). TLRs, RAGE-R, DNGR (encoded by Clec9A), and Mincle have been shown to be receptors responsible for mediating inflammation initiated by a variety of DAMPs.

The inventors' recent work demonstrated that CD24-Siglec G interactions discriminate innate immunity to DAMPs from PAMPs. Siglec proteins are membrane-associated immunoglobulin (Ig) superfamily members that recognize a variety of sialic acid-containing structures. Most Siglecs have an intra-cellular immune-tyrosine inhibitory motif (ITIM) that associates with SHP-1, -2 and Cb1-b to control key regulators of inflammatory responses. The inventors have reported CD24 as the first natural ligand for a Siglec, Siglec G in mouse and Siglec 10 in human. Siglec G interacts with sialylated CD24 to suppress the TLR-mediated host response to DAMPs, such as HMGB1, via a SHP-1/2 signaling mechanism.

Human CD24 is a small glycoslphosphatidylinositol (GPI)-anchored protein encoded by an open-reading frame of 240 nucleotide base pairs in the CD24 gene. Of the 80 amino acids, the first 26 amino acids at the NH₂-terminus of the protein constitute the signal peptide, while the last 23 amino acids at the COOH-terminus serve as a signal for cleavage to allow for the attachment of the GPI tail. As a result, the mature human CD24 polypeptide has only 31 amino acids, which also represents the extracellular domain of the human CD24 protein. One of the 31 amino acids is polymorphic among the human population. A cytosine (C) to thymine (T) nucleotide transition at nucleotide 170 of the CD24 gene open-reading frame results in the amino acid substitution of alanine (A) with valine (V) [at amino acid residue 31 of the mature CD24 protein]. Since this amino acid residue is positioned immediately N-terminal to the GPI signal cleavage site, and since the replacement is nonconservative, these two alleles may be expressed at different efficiencies on the cell surface. Indeed, transfection studies with cDNA demonstrated that the CD24^{v} allele (containing the valine residue) is more efficiently expressed on the cell surface. Consistent with this, CD24^{v/v} PBLexpressed higher levels of CD24, especially on T cells.

The inventors have demonstrated that CD24 negatively regulates host response to cellular DAMPs that are released as a result of tissue or organ damage, and at least two overlapping mechanisms may explain this activity (Chen, G. Y., et al., CD24 and Siglec-10 selectively repress tissue damage-induced immune responses, Science, Vol. 323, pp. 1722-1725 (2009)). First, CD24 binds to several DAMPs, including HSP70, HSP90, HMGB1 and nucleolin and represses host response to these DAMPs. To do this, it is presumed that CD24 may trap the inflammatory stimuli to prevent interaction with their receptors, TLR or RAGE. Second, using an acetaminophen-induced mouse model of liver necrosis and ensuring inflammation, the inventors demonstrated that through interaction with its receptor, Siglec G, CD24 provides a powerful negative regulation for host response to tissue injuries. To achieve this activity, CD24 may bind and stimulate signaling by Siglec G wherein Siglec G-associated SHP1 triggers the negative regulation. Both mechanisms may act in concert as mice with targeted mutation of either gene mounted much stronger inflammatory response. In fact, dendritic cells cultured from bone marrow from either CD24^{-/-} or Siglec G^{-/-} mice produced higher levels of inflammatory cytokines when stimulated with either HMGB1, HSP70, or HSP90. CD24 protein appears to be the only inhibitory DAMP receptor capable of shutting down inflammation triggered by DAMPs and no drug is currently available that specifically targets host inflammatory response to tissue injuries. Furthermore, the inventors have demonstrated the ability of exogenous soluble CD24 protein to alleviate DAMP-mediated autoimmune disease using mouse models of RA, multiple sclerosis (MS) and graft versus host disease (GvHD).

As CD24Fc is an agonist of Siglecs and can fortify the CD24-Siglec innate immune checkpoint, the inventors evaluated whether CD24Fc could improve the lung pathology of SIV-infected rhesus monkeys that received either placebo (Normal saline, NS) or CD24Fc. In particular, they demonstrated that CD24-Siglec pathway can protect against destructive inflammation triggered by cell death.

In an embodiment of the invention, the CD24 protein administered by the methods described herein may comprise a mature human CD24 polypeptide, which constitutes the extracellular domain (ECD) of the CD24 protein, or a variant thereof. The mature human CD24 polypeptide or variant thereof is represented by SEQ ID NO: 1 or 2. The CD24 protein may comprise a protein tag, which may be fused at the N-terminus or C-terminus of the CD24 protein. The protein tag may comprise a portion of a mammalian immunoglobulin (Ig) protein, and the portion may be a Fc region. The Ig protein may be human. The Fc region may comprise a hinge region and CH2 and CH3 domains of a human Ig protein selected from the group consisting of IgG1, IgG2, IgG3, IgG4, and IgA. The Fc region may comprise a hinge region and CH2, CH3, and CH4 domains of IgM. The amino acid sequence of the CD24 protein may comprise or consist of a sequence set forth in SEQ ID NO: 6, 11, or 12.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

A “peptide” or “polypeptide” is a linked sequence of amino acids and may be natural, synthetic, or a modification or combination of natural and synthetic.

“Substantially identical” may mean that a first and second amino acid sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, or 300 amino acids.

“Treatment” or “treating,” when referring to protection of an animal from a disease, means preventing, suppressing, repressing, or completely eliminating the disease and/or infection. Preventing the disease involves administering a composition of the present invention to an animal prior to onset of the disease. Suppressing the disease involves administering a composition of the present invention to an animal after induction of the disease but before its clinical appearance. Repressing the disease involves administering a composition of the present invention to an animal after clinical appearance of the disease.

A “variant” may mean a peptide or polypeptide that differs in amino acid sequence by the insertion, deletion, or conservative substitution of amino acids, but retain at least one biological activity. Representative examples of “biological activity” include the ability to bind to a toll-like receptor and to be bound by a specific antibody. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

Mature human CD24 protein as used herein, is also sometimes referred to as mature human CD24 polypeptide and as human CD24 ECD.

An amino acid modification, as used herein, refers to a substitution of an amino acid, including substitution with any of the 20 amino acids commonly found in human proteins or with an atypical or non-naturally occurring amino acid, or the derivation of an amino acid by the addition and/or removal of chemical groups to/from the amino acid. Commercial sources of atypical amino acids include Sigma-Aldrich (Milwaukee, Wis.), ChemPep Inc. (Miami, Fla.), and Genzyme Pharmaceuticals (Cambridge, Mass.). Atypical amino acids may be purchased from commercial suppliers, synthesized de novo, or chemically modified or derivatized from naturally occurring amino acids.

An amino acid substitution, as used herein, refers to the replacement of one amino acid residue by a different amino acid residue (including an atypical or non-naturally occurring amino acid).

A conservative amino acid substitution, as used herein, is defined as exchanges within one of the following five groups of amino acids:

I. Small aliphatic, nonpolar or slightly polar residues: Ala, Ser, Thr, Pro, Gly;

II. Polar, negatively charged residues and their amides: Asp, Asn, Glu, Gln, cysteic acid and homocysteic acid;

III. Polar, positively charged residues: His, Arg, Lys; Ornithine (Orn);

IV. Large, aliphatic, nonpolar residues: Met, Leu, Ile, Val, Cys, Norleucine (Nle), homocysteine;

V. Large, aromatic residues: Phe, Tyr, Trp, acetyl phenylalanine.

### 2. CD24

Provided herein are methods that include administering a CD24 protein. The CD24 protein may comprise a mature CD24 polypeptide or a variant thereof. The mature form of the CD24 protein corresponds to the extracellular domain (ECD). The mature CD24 protein administered as part of the invention may be derived from a human or another mammal. As described above, mature human CD24 protein is 31 amino acids long and has a variable alanine (A) or valine (V) residue at its C-terminal end, as follows:

(SEQ ID NO: 1)
SETTTGTSSNSSQSTSNSGLAPNPTNATTK(V/A)

The C-terminal valine or alanine as shown in SEQ ID NO: 1 may be immunogenic and when omitted from the CD24 protein provided herein, may reduce its immunogenicity. Therefore, the CD24 protein provided herein may comprise the amino acid sequence of mature human CD24 lacking the C-terminal valine or alanine amino acid, as follows:

(SEQ ID NO: 2)
SETTTGTSSNSSQSTSNSGLAPNPTNATTK

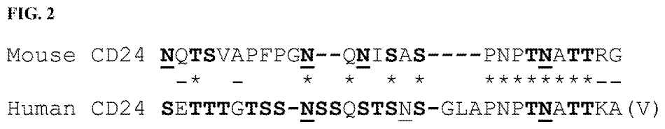

Despite considerable sequence variations in the amino acid sequence of the extracellular domain of mature CD24 proteins from mouse and human, they are functionally equivalent, as a soluble form of human CD24 has been shown to be active in the mouse. The amino acid sequence of the human CD24 ECD (SEQ ID NO: 1) is 39% identical to the corresponding mouse protein (Genbank accession number NP 033976). However, it is not that surprising that the percent identity is not higher as the CD24 ECD is only 27-31 amino acids in length, depending on the species, and binding to some of its receptor(s), such as Siglec 10/G, is mediated by its sialic acid and/or galactose sugars of the glycoprotein. The amino acid sequence identity between the extracellular domains of the human Siglec-10 (GenBank accession number AF310233) and its murine homolog Siglec-G (GenBank accession number NP_766488) receptor proteins is 63% (FIG. 2). Since the sequence conservation between the mouse and human mature CD24 proteins is primarily in the C-terminus of the proteins and there is an abundance of potential glycosylation sites (S and T residues) in the extracellular domain, the CD24 proteins provided herein may tolerate significant variations from the mature CD24 sequence as shown in SEQ ID NO: 1, especially if those variations do not affect the conserved residues in the C-terminus and/or the glycosylation sites.

Therefore, the CD24 protein provided herein may comprise the amino acid sequence of mature murine CD24:

(SEQ ID NO: 3)
NQTSVAPFPGNQNISASPNPTNATTRG

The amino acid sequence of the human CD24 ECD shows more sequence conservation with the cynomolgus monkey version of the protein (52% identity; UniProt accession number UniProtKB-I7GKK1) than with mouse. Again, it is not surprising given that the percent identity is not higher as the ECD is only 29-31 amino acids in length in these species, and the role of sugar residues in binding to its receptor(s). The amino acid sequence of cynomolgous Siglec-10 receptor has not been determined, but the amino acid sequence identity between the human and rhesus monkey Siglec-10 (GenBank accession number XP_001116352) proteins is 89%. Therefore, the CD24 protein provided herein may also comprise the amino acid sequence of mature cynomolgous (or rhesus) monkey CD24:

(SEQ ID NO: 10)
TVTTSAPLSSNSPQNTSTTPNPANTTTKA

The CD24 protein administered as part of the disclosed methods may be soluble. The CD24 protein may further comprise an N-terminal signal peptide, to allow secretion from a cell expressing the protein. The signal peptide sequence may comprise the amino acid sequence:

(SEQ ID NO: 4)
MGRAMVARLGLGLLLLALLLPTQIYS

Alternatively, the signal sequence may be any of those that are found on other transmembrane or secreted proteins, or those modified from the existing signal peptides known in the art.

a. Fusion

The CD24 protein administered by the methods described herein may be fused at its N- or C-terminal end to a protein tag, which may comprise a portion of a mammalian Ig protein, which may be human or mouse or from another species. The portion may comprise an Fc region of the Ig protein. The Fc region may comprise at least one of the hinge region, CH2, CH3, and CH4 domains of the Ig protein. The Ig protein may be human IgG1, IgG2, IgG3, IgG4, or IgA, and the Fc region may comprise the hinge region, and CH2 and CH3 domains of the Ig. The Fc region may comprise the human immunoglobulin G1 (IgG1) isotype SEQ ID NO: 7. The Ig protein may also be IgM, and the Fc region may comprise the hinge region and CH2, CH3, and CH4 domains of IgM. The protein tag may be an affinity tag that aids in the purification of the protein, and/or a solubility-enhancing tag that enhances the solubility and recovery of functional proteins. The protein tag may also increase the valency of the CD24 protein. The protein tag may also comprise GST, His, FLAG, Myc, MBP, NusA, thioredoxin (TRX), small ubiquitin-like modifier (SUMO), ubiquitin (Ub), albumin, or a Camelid Ig. Methods for making fusion proteins and purifying fusion proteins are well known in the art.

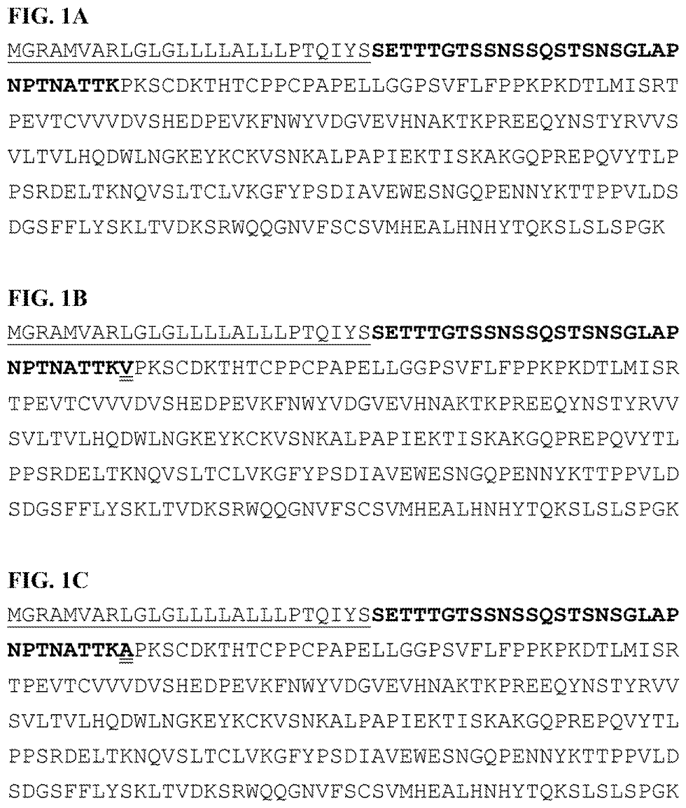

For the construction of the fusion protein CD24Fc identified in the examples, a truncated form of the mature CD24 ECD molecule of 30 amino acids as represented by SEQ ID NO: 2, lacking the final (C-terminal) polymorphic amino acid of the mature human CD24 protein (located before the GPI signal cleavage site of the full-length CD24 protein), has been used. This variant of the mature human CD24 ECD sequence is fused to a human IgG1 Fc domain represented by SEQ ID NO: 7. A “full-length” version of the CD24Fc fusion protein is provided in SEQ ID NO: 5 (FIG. 1A), which contains the 30 amino acid mature CD24 variant protein and the CD24 signal peptide fused to the IgG1 Fc domain at the C-terminus of the CD24 polypeptide. A processed version of the full-length CD24Fc fusion protein that is secreted from the cell (i.e., lacking the signal sequence, which is cleaved off) is provided in SEQ ID NO: 6, which contains just the 30 amino acid mature CD24 variant polypeptide fused to the IgG1 Fc domain. Processed polymorphic variants of mature CD24 polypeptide (that is, mature CD24 polypeptide having SEQ ID NO: 1) fused to IgG1 Fc may comprise the amino acid sequence set forth in SEQ ID NO: 11 or 12.

Thus, as used herein, “CD24Fc” is a fusion that corresponds to a variant of the mature human CD24 protein wherein the last (C-terminal) amino acid (A or V) of the mature human CD24 protein has been deleted, and wherein the mature human CD24 polypeptide variant is fused to an IgG1 Fc domain. SEQ ID NO: 6 is an example of such a CD24 fusion protein, wherein the IgG1 Fc domain is fused to the C-terminal of the human mature CD24 polypeptide variant.

b. Production

The CD24 protein administered by the methods described herein, which includes Fc fusion proteins of CD24 as described, may be heavily glycosylated, and may be involved in functions of CD24 protein such as costimulation of immune cells and interaction with a damage-associated molecular pattern molecule (DAMP). The CD24 protein may be prepared using a eukaryotic expression system. The expression system may entail expression from a vector in mammalian cells, such as Chinese Hamster Ovary (CHO) cells. The system may also be a viral vector, such as a replication-defective retroviral vector that may be used to infect eukaryotic cells. The CD24 protein may also be produced from a stable cell line that expresses the CD24 protein from a vector or a portion of a vector that has been integrated into the cellular genome. The stable cell line may express the CD24 protein from an integrated replication-defective retroviral vector. The expression system may be GPEX™ (Catalent Biotechnology, Somerset, N.J.).

c. Pharmaceutical Composition

The CD24 protein administered by the methods described herein, which includes Fc fusion proteins of CD24 as described, may be contained in a pharmaceutical composition, which may comprise a pharmaceutically acceptable amount of the CD24 protein. The pharmaceutical composition may comprise a pharmaceutically acceptable carrier. The pharmaceutical composition may comprise a solvent, which may keep the CD24 protein stable over an extended period. The solvent may be PBS, which may keep the CD24 protein stable for at least 66 months at −20° C. (−15˜−25° C.). The solvent may be capable of accommodating the CD24 protein in combination with another drug.

The pharmaceutical composition may be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water. In one example, the pharmaceutical composition comprises a normal saline solution, in which the CD24 protein may be diluted. The volume of the saline solution may be 100 mL. In one example, the pharmaceutical composition for intravenous administration comprises 480 mg of the CD24 protein, including Fc fusion proteins of mature human CD24 as described.

The pharmaceutical composition may also be formulated as a depot preparation, which may be administered by implantation or by intramuscular injection. The composition may be formulated with suitable polymeric or hydrophobic materials (as an emulsion in an acceptable oil, for example), ion exchange resins, or as sparingly soluble derivatives (as a sparingly soluble salt, for example).

The dose of the CD24 protein administered by the method described herein, which includes Fc fusion proteins of mature CD24 as described, may ultimately be determined through a clinical trial to determine a dose with acceptable toxicity and clinical efficacy. The initial clinical dose may be estimated through pharmacokinetics and toxicity studies in rodents and non-human primates. The dose of the CD24 protein may be 0.01 mg/kg to 1000 mg/kg, and may be 1 to 500 mg/kg, depending on the desired effect on irAEs or GvHD and the route of administration. The CD24 protein may be administered by intravenous infusion or subcutaneous, intramural (that is, within the wall of a cavity or organ), or intraperitoneal injection, and the dose may be 10-1000 mg, 10-500 mg, 10-240 mg, 10-120 mg, or 10, 30, 60, 120, 240 mg, or 480 mg, where the subject is a human.

### 3. Methods of Treatment

a. Pneumonia

Viral pneumonia accounts for more than ⅓ of pneumonia in human and is thus a major cause of mortality. The pneumonia may be caused by the primary SARS, including SARS-CoV, SARS-CoV2 infection or be caused in the SARS-infected patient by a secondary bacterial infection, which may follow the SARS viral infection. In particular, Staphylococci are the most frequent cause of secondary pneumonia. Most recently, severe pneumonia can be induced by coronavirus infection, including with SARS-CoV, SARS-CoV-2 and/or MERS. Accordingly, the CD24 protein administered by the methods described herein, which includes Fc fusion proteins of CD24 as described, may be used to treat or prevent severe pneumonia induced by coronaviruses, including SARS-CoV, SARS-CoV-2 or MERS, in a subject in need thereof. The CD24 protein used to treat or prevent severe pneumonia induced by coronavirus, including SARS-CoV, SARS-CoV-2 or MERS, may comprise a mature human CD24 polypeptide or a variant thereof, as illustrated in the sequences set forth in SEQ ID NO: 1 or 2. The CD24 protein used to treat or prevent severe pneumonia induced by coronavirus, including SARS-CoV, SARS-CoV-2 or MERS, may also comprise a protein tag, wherein the protein tag is fused at the N-terminus or C-terminus of the mature CD24 protein or variant thereof. The protein tag may comprise a portion of a mammalian immunoglobulin (Ig) protein, wherein the portion of the mammalian Ig protein may be a Fc region. The Ig protein may be human. The Fc region may comprise a hinge region and CH₂ and CH₃ domains of a human Ig protein selected from the group consisting of IgG1, IgG2, IgG3, IgG4, and IgA. The Fc region may comprise a hinge region and CH2, CH3 and CH4 domains of IgM. The amino acid sequence of the CD24 protein used to treat or prevent severe pneumonia induced by coronavirus, including SARS-CoV, SARS-CoV-2 or MERS, may comprise a sequence set forth in SEQ ID NO: 6, 11, or 12. The amino acid sequence of the CD24 protein used to treat or prevent severe pneumonia induced by coronavirus, including SARS-CoV, SARS-CoV-2 or MERS, may comprise a sequence set forth in SEQ ID NO: 6. The amino acid sequence of the CD24 protein used to treat or prevent severe pneumonia induced by coronavirus, including SARS-CoV, SARS-CoV-2 or MERS, may comprise a sequence set forth in SEQ ID NO: 11. The amino acid sequence of the CD24 protein used to treat or prevent severe pneumonia induced by coronavirus, including SARS-CoV, SARS-CoV-2 or MERS, may comprise a sequence set forth in SEQ ID NO: 12. The amino acid sequence of the CD24 protein used to treat or prevent severe pneumonia induced by coronavirus, including SARS-CoV, SARS-CoV-2 or MERS, may also consist of a sequence set forth in SEQ ID NO: 6, 11, or 12. The CD24 protein used to treat or prevent severe pneumonia induced by coronavirus, including SARS-CoV, SARS-CoV-2 or MERS, can be produced using a eukaryotic protein expression system wherein the expression system may comprise a vector contained in a Chinese Hamster Ovary cell line or a replication-defective retroviral vector and wherein the replication-defective retroviral vector is stably integrated into the genome of a eukaryotic cell. The CD24 protein used to treat or prevent severe pneumonia induced by coronavirus, including SARS-CoV, SARS-CoV-2 or MERS, is soluble and glycosylated. An embodiment includes the use of a CD24 protein as described herein in the manufacture of a medicament for use to treat or prevent severe pneumonia induced by coronavirus, including SARS-CoV, SARS-CoV-2 or MERS, in a subject.

In particular, a CD24 protein as disclosed herein may be used to treat a SARS-CoV-2 infection. The subject may have or been diagnosed with COVID-19 disease. In one example, the subject may be hospitalized and/or may require oxygen therapy (for example, one or more of supplemental oxygen, non-invasive mechanical ventilation, and a high flow oxygen device). The subject may also have a NIAID 8-point ordinal score 3-4 (described in Table 2, Example 5), which may be regardless of acute respiratory distress syndrome (ARDS).

The subject may be a mammal. In particular, the mammal may be a monkey or an ape. The ape may be a chimpanzee, gorilla, or human. The human may be a man, a woman, White, Black or African-American, Asian, Hispanic or Latino, or Non-Hispanic or Latino.

Administering the CD24 protein as disclosed herein may result in an improvement in clinical status, as measured by the MAID 8-point ordinal scale, which may be within 28 days of administering the CD24 protein. In one example, the subject improves from scales 3-4 to ≥5, and may additionally not drop below scale 5 within 28 days of administering the CD24 protein. Administering the CD24 protein may result in reduced time to clinical improvement, as measured by the time in days required from administering the CD24 protein to the improvement of clinical status from “scale 3 or 4” to “scale 5 or higher” based on the MAID 8-point ordinal scale within 28 days from randomization.

Administering the CD24 protein as disclosed herein may result in one or more of: a reduction in the risk of death or respiratory failure, defined as the need for mechanical ventilation, extracorporeal membrane oxygenation, non-invasive ventilation, or high flow oxygen devices, within 28 days from administering the CD24 protein; time to disease progression (TTDP), defined as the time from administering the CD24 protein to invasive mechanical ventilation or death; and the risk of death from any cause within 29 days of administering the CD24 protein. One or more changes in a subject's characteristics described herein resulting from administering the CD24 protein may be as compared to a population of patients treated with a placebo and/or who received standard of care for a SARS-CoV-2 infection and/or Covid-19.

The treatment may result in one or more of: a reduced risk of death; a reduced duration of treatment with mechanical ventilation, a reduced duration of treatment with extracorporeal membrane oxygenation, a reduced duration of treatment with non-invasive ventilation, a reduced duration of treatment with pressors, a reduced duration of treatment with a high flow oxygen device, a decreased rate of disease progression, an increase time to clinical relapse, a reduced duration of supplemental oxygen, a reduced time of hospital stay, a reduced absolute lymphocyte count, reduced levels of one or more markers of inflammation, and decreased D-dimer concentration in plasma. Disease progression may be measured according to the MAID 8-point ordinal scale, such as progression from scale 3 or 4 to scale 1 or 2, or 2 to 1, and which may be measured within 28 days from administering the CD24 protein. In one example, the treatment may reduce the risk of respiratory failure (such as the need for mechanical ventilation, extracorporeal membrane oxygenation, non-invasive ventilation, or high flow oxygen device), which may be within 28 days of administering the CD24 protein. The treatment may also decrease the time to disease progress (such as invasive mechanical ventilation or death).

In both primary and secondary pneumonia, viruses and bacteria may cause death of infected cells, which in turn can trigger inflammation in the lung. Therefore, therapeutics that suppress tissue injury-induced inflammation may also be used to treat viral and bacterial pneumonia.

b. Administration

The route of administration of the CD24 protein pharmaceutical compositions described herein may be parenteral. Parenteral administration includes, but is not limited to, intravenous, intraarterial, intraperitoneal, subcutaneous, intramuscular, intrathecal, intraarticular, and direct injection. The composition may be administered 1, 2, 3, 4, 5, 6, 7, 8, 9, 10, 11, or 12 times per day. The composition may be administered for a period of 1, 2, 3, 4, 5, 6, 7, 8, 9, 10, 11, 12, 13, or 14 days, or 3, 4, 5, 6, 7, 8, 9, 10, 11, or 12 weeks.

In one example, the CD24 protein described herein may be administered at a 480 mg dose. In one embodiment, the CD24 protein may be infused intravenously, which may be administered over a period of up to 30, 40, 50, 60, 70, 80, 90, 100, 110, or 120 minutes, or up to 3, 4, 5, 6, 7 or 8 hours. The CD24 protein may be diluted in normal saline for intravenous administration, including for example to a 100 mL volume.

c. Combination Treatment

Since viral and bacterial infections are the primary cause of viral pneumonia, antivirals and antibiotics are frequently used as the treatment of choice. However, since pneumonia are lung inflammation that can be caused by tissue injuries following infection, other treatment can be used in combination with reagents that primarily inhibit infections, such reagents includes commonly used antivirals as well as antibiotics as indicated by specific pathogens.

In one embodiment, treatment with the CD24 protein as described herein may be combined with standard of care for a SARS-CoV-2 infection. The CD24 protein may also be combined with one or more other treatments of the infection, including for example oxygen therapy, mechanical ventilation, extracorporeal membrane oxygenation, non-invasive ventilation, and a high flow oxygen device. Treatment with the CD24 protein may also be combined with one or more of an antiviral agent (for example, remdesivir, or molnupiravir), a corticosteroid (for example, dexamethasone, prednisone, or methyl prednisolone), and an immune modulator (for example, an IL-6/IL-6R antagonist). In one example, the subject may be treated with remdesivir in combination with the CD24 protein. In another example, the subject may be treated with molnupiravir in combination with the CD24 protein.

The CD24 protein as described herein may be administered simultaneously or metronomically with other treatments. The term “simultaneous” or “simultaneously” as used herein, means that the CD24 protein and other treatment be administered within 48 hours, preferably 24 hours, more preferably 12 hours, yet more preferably 6 hours, and most preferably 3 hours or less, of each other. The term “metronomically” as used herein means the administration of the agent at times different from the other treatment and at a certain frequency relative to repeat administration.

The CD24 protein as described herein may be administered at any point prior to another treatment including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins, 10 mins, 9 mins, 8 mins, 7 mins., 6 mins., 5 mins., 4 mins., 3 mins, 2 mins, or 1 min. The CD24 protein may be administered at any point prior to a second treatment of the CD24 protein including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins., 10 mins., 9 mins., 8 mins., 7 mins., 6 mins., 5 mins., 4 mins., 3 mins, 2 mins, or 1 min.

The CD24 protein as described herein may be administered at any point after another treatment including about 1 min., 2 mins., 3 mins., 4 mins., 5 mins., 6 mins., 7 mins., 8 mins., 9 mins., 10 mins., 15 mins., 20 mins., 25 mins., 30 mins., 35 mins., 40 mins., 45 mins., 50 mins., 55 mins., 1 hr, 2 hr, 3 hr, 4 hr, 6 hr, 8 hr, 10 hr, 12 hr, 14 hr, 16 hr, 18 hr, 20 hr, 22 hr, 24 hr, 26 hr, 28 hr, 30 hr, 32 hr, 34 hr, 36 hr, 38 hr, 40 hr, 42 hr, 44 hr, 46 hr, 48 hr, 50 hr, 52 hr, 54 hr, 56 hr, 58 hr, 60 hr, 62 hr, 64 hr, 66 hr, 68 hr, 70 hr, 72 hr, 74 hr, 76 hr, 78 hr, 80 hr, 82 hr, 84 hr, 86 hr, 88 hr, 90 hr, 92 hr, 94 hr, 96 hr, 98 hr, 100 hr, 102 hr, 104 hr, 106 hr, 108 hr, 110 hr, 112 hr, 114 hr, 116 hr, 118 hr, or 120 hr. The CD24 protein may be administered at any point after a previous CD24 treatment including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins., 10 mins., 9 mins., 8 mins., 7 mins., 6 mins., 5 mins., 4 mins., 3 mins, 2 mins, or 1 min.

### Example 1

CD24 Pharmacokinetics in Mice

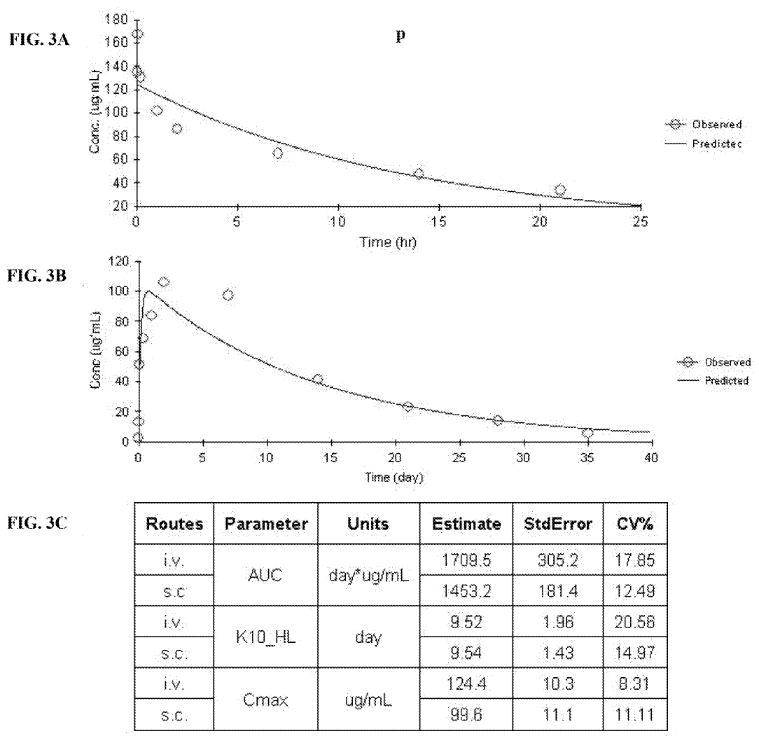

1 mg of CD24Fc represented by SEQ ID NO. 6 was injected into naïve C57BL/6 mice and blood samples were collected at different timepoints (5 min, 1 hr, 4 hrs, 24 hrs, 48 hrs, 7 days, 14 days and 21 days) from 3 mice in each timepoint. The sera were diluted 1:100 and the levels of CD24Fc was detected using a sandwich ELISA using purified anti-human CD24 (3.3 μg/ml) as the capturing antibody and peroxidase conjugated goat anti-human IgG Fc (5 μg/ml) as the detecting antibodies. As shown in FIG. 3A, the decay curve of CD24Fc revealed a typical biphasic decay of the protein. The first biodistribution phase had a half-life of 12.4 hours. The second phase follows a model of first-order elimination from the central compartment. The half-life for the second phase was 9.54 days, which is similar to that of antibodies in vivo. These data suggest that the fusion protein is very stable in the blood stream. In another study in which the fusion protein was injected subcutaneously, an almost identical half-life of 9.52 days was observed (FIG. 3B). More importantly, while it took approximately 48 hours for the CD24Fc to reach peak levels in the blood, the total amount of the fusion protein in the blood, as measured by AUC, was substantially the same by either route of injection. Thus, from a therapeutic point of view, using a different route of injection should not affect the therapeutic effect of the drug. This observation greatly simplified the experimental design for primate toxicity and clinical trials.

### Example 2

CD24-Siglec 10 Interaction in Host Response to Tissue Injuries

Nearly two decades ago, Matzinger proposed what was popularly called danger theory (P. Matzinger, “Tolerance, danger, and the extended family,” Annual Review of Immunology, vol. 12, pp. 991-1045, 1994). In essence, she argued that the immune system is turned on when it senses the dangers in the host. Although the nature of danger was not well defined at the time, it has been determined that necrosis is associated with the release of intracellular components such as HMGB1 and Heat-shock proteins, which were called DAMP, for danger-associated molecular patterns. DAMP were found to promote production of inflammatory cytokines and autoimmune diseases. In animal models, inhibitors of HMGB1 and HSP90 were found to ameliorate rheumatoid arthritis (RA). The involvement of DAMP raised the prospect that negative regulation for host response to DAMP can be explored for RA therapy.

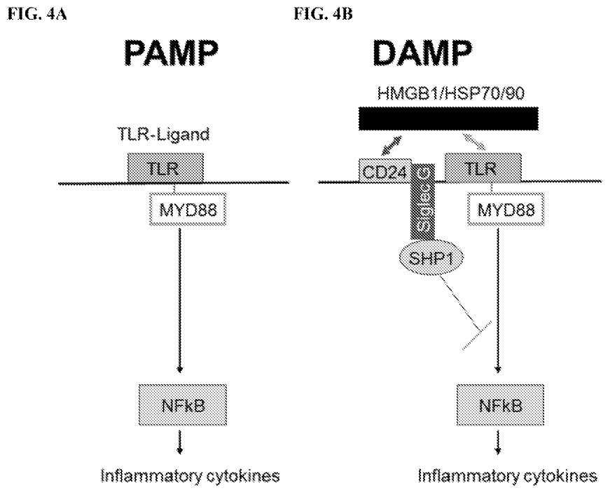

Using acetaminophen-induced liver necrosis and ensuring inflammation, it was observed that through interaction (with) Siglec G, CD24 provides a powerful negative regulation for host response to tissue injuries. CD24 is a GPI anchored molecule that is broadly expressed in hematopoietic cells and other tissue stem cells. Genetic analysis of a variety of autoimmune disease in human, including multiple sclerosis, systemic lupus erythromatosus, RA, and giant cell arthritis, showed significant association between CD24 polymorphism and risk of autoimmune diseases. Siglec G is a member of I-lectin family, defined by their ability to recognize sialic acid containing structure. Siglec G recognizes sialic acid containing structure on CD24 and negatively regulates production of inflammatory cytokines by dendritic cells (DC). In terms of its ability to interact with CD24, human Siglec 10 and mouse Siglec G are functionally equivalent. However, it is unclear if there is a one-to-one correlation between mouse and human homologues. Although the mechanism remains to be fully elucidated, it is plausible that SiglecG-associated SHP1 may be involved in the negative regulation. These data lead to a new model in which CD24-Siglec G/10 interaction may play a critical role in discrimination pathogen-associated molecular pattern (PAMP) from DAMP (FIG. 4).

At least two overlapping mechanisms may explain the function of CD24. First, by binding to a variety of DAMP, CD24 may trap the inflammatory stimuli to prevent their interaction with TLR or RAGE. This notion is supported by observations that CD24 is associated with several DAMP molecules, including HSP70, 90, HMGB1 and nucleolin. Second, perhaps after associated with DAMP, CD24 may stimulate signaling by Siglec G. Both mechanisms may act in concert as mice with targeted mutation of either gene mounted much stronger inflammatory response. In fact, DC cultured from bone marrow from either CD24^{-/-} or Siglec G^{-/-} mice produced much higher inflammatory cytokines when stimulated with either HMGB1, HSP70, or HSP90. In contrast, no effect were found in their response to PAMP, such as LPS and PolyI:C. These data not only provided a mechanism for the innate immune system to distinguish pathogen from tissue injury, but also suggest CD24 and Siglec G as potential therapeutic targets for diseases associated with tissue injuries. Although CD24-Siglec interaction does not control infection, it can also affect inflammatory response during viral and bacterial infection. For instance, many infections cause tissue injury. In addition, many infectious agents may disrupt CD24-Siglec interaction, as they encode sialidase that removes sialic acid from CD24 and thereby inactivate the negative regulation of inflammation caused by tissue injuries.

### Example 3

CD24Fc Interacts with HMGB1, Siglec 10 and Induces Association Between Siglec G and SHP-1

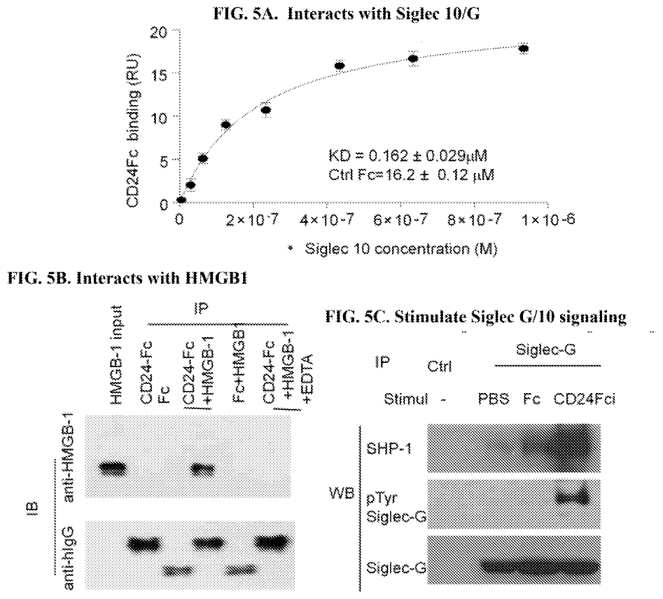

To measure the interaction between CD24Fc and Siglec 10, we immobilized CD24Fc onto a CHIP and used Biacore to measure the binding of different concentrations of Siglec-10Fc. As shown in FIG. 5A, CD24Fc binds with Siglec 10 with a Kd of 1.6×10⁻⁷M. This is 100-fold higher affinity than the control Fc. The interaction between CD24Fc and HMGB1 was confirmed by pull down experiments using CD24Fc-bound protein G beads followed by Western blot with either anti-IgG or anti-HMGB1. These data demonstrate that CD24Fc, but not Fc, binds to HMGB1 and that this binding is cation-dependent (FIG. 5B). To determine whether CD24Fc is an agonist of Siglec G, the mouse counterpart of human Siglec 10, we stimulated CD24^{-/-} spleen cells with CD24Fc, control Fc or vehicle (PBS) control for 30 minutes. Siglec G was then immunoprecipitated and probed with anti-phospho-tyrosine or anti-SHP-1. As shown in FIG. 5C, CD24Fc induced substantial phosphorylation of Siglec G and association of SHP-1, a well-known inhibitor for both adaptive and innate immunity.

In Vitro Efficacy Studies of CD24Fc.

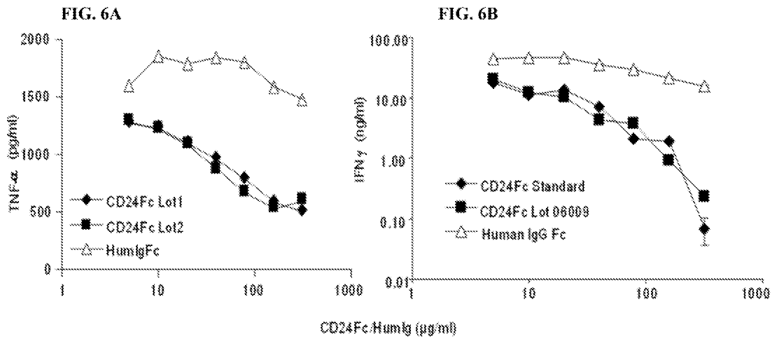

To study the impact of CD24Fc on the production of inflammatory cytokines by human T cells, the mature T cells in human PBML were activated by anti-CD3 antibody (OKT3), a commonly used agonist of the T cell receptor in the presence of different concentrations of CD24Fc or human IgG1 Fc. Four days later, the supernatants were collected and the production of IFN-γ and TNF-α were measured by Enzyme-linked immunosorbent assay (ELISA) to confirm activation. The results in FIG. 6 demonstrated that CD24Fc from two different manufacturing lots significantly reduced IFN-γ and TNF-α production from the activated human PBML compared with control IgG Fc control. In addition, when CD24Fc was added, cytokine production was inhibited in a dose-dependent manner. Therefore, CD24Fc can inhibit anti-CD3 induced human PBML activation in vitro. This study not only indicated the mechanism of action of CD24Fc might be through the inhibition of T cell activation, but also established a reliable bioassay for drug potency and stability testing.

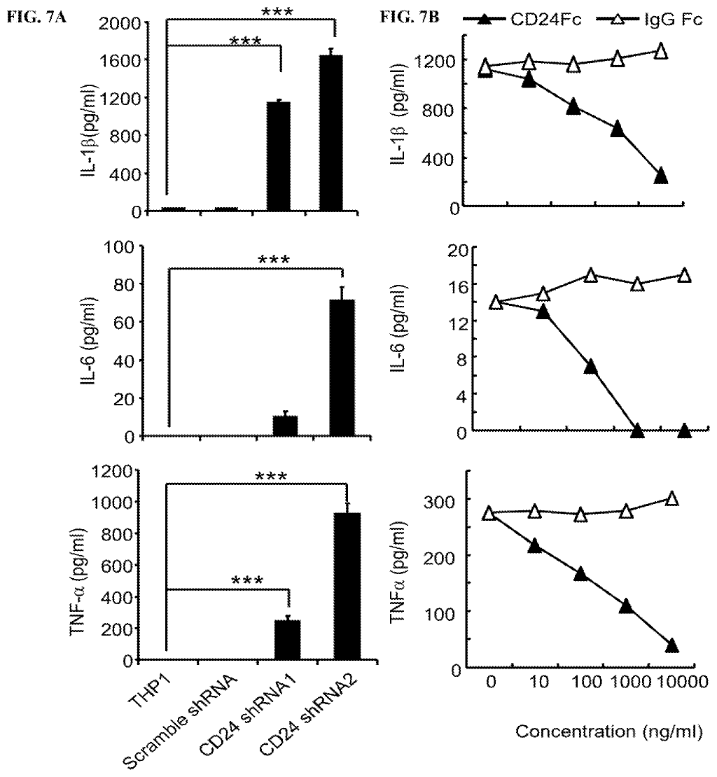

To determine whether CD24Fc regulates production of inflammatory cytokines in a human cell line, we first silenced CD24 in the human acute monocytic leukemia THP1 cell line using RNAi, and then induced differentiation into macrophages by treating them with PMA. As shown in FIG. 7A, CD24 silencing substantially increased the production of TNFα, IL-1β and IL-6. These data demonstrate an essential role for endogenous human CD24 in limiting the production of inflammatory cytokines. Importantly, CD24Fc restored inhibition of TNFα in the CD24-silenced cell line (FIG. 7B), as well as IL-1 β and IL-6. These data not only demonstrate the relevance of CD24 in inflammatory response of human cells, but also provides a simple assay to assess biological activity of CD24Fc.

Taken together, these data demonstrate that CD24Fc is capable of inhibiting cytokine production triggered by adaptive and innate stimuli. However, since the drug is much more effective in reducing cytokine production by innate effectors, we consider that the primary mechanism for its prophylactic function is to prevent inflammation triggered by tissue injuries at the early phase of transplantation.

### Example 4

CD24 for Treating a SARS-CoV-2 Infection

This example demonstrates that CD24Fc described herein is capable of treating a SARS-CoV-2 infection.

### SUMMARY

A Phase III study is being conducted. It is a randomized double-blind placebo controlled study with planned enrollment of 270 subjects. Patients are adults (≥18 years old) with confirmed SARS-CoV-2 viral infection and hospitalized requiring oxygen therapy, including supplemental oxygen, non-invasive mechanical ventilation or high flow oxygen devices. Patients are randomized 1:1 to receive one of the following treatments as single dose administration on Day 1.

Arm A: CD24Fc, 480 mg, diluted to 100 ml with normal saline, IV infusion in 60 minutes.

Arm B: placebo, normal saline 100 ml, IV infusion in 60 minutes.

The best available treatment and supportive care is given to all subjects according to local institutional guideline.

The study had the first patient enrolled on Apr. 24, 2020. The last patient enrolled on Aug. 21, 2020 for this interim Data and Safety Monitoring Board (DSMB) report. The accrual period is 119 days.

As of the cutoff date of Aug. 27, 2020, there were 203 subjects enrolled in the study, with 197 subject randomized and 6 screen failures. There were 194 subjects who completed the treatment of a single dose of CD24Fc/placebo treatment. Three subjects were randomized but did not receive treatment. 99 subjects were in CD24Fc treatment arm, and 98 subjects were in placebo arm.

There were no abnormal changes in vital signs recorded at timepoints of pre-dose, 15 min, 30 min, 60 min and 120 min. There were no abnormal changes in ECG recorded at timepoints of pre-dose and 2 hours after dosing. There were no infusion reactions or delayed allergic reactions to the treatment. There was no discontinuation of treatment due to adverse events. The treatment was well tolerated to all 194 subjects.

Mortality: There were 26 subjects who expired within 28 days from randomization. 11 subjects were from the CD24Fc treatment arm, and 15 subjects were from the placebo arm. Additional 4 subjects expired beyond 28 days after randomization. One subject was from the CD24Fc treatment arm, and 3 subjects were from the placebo arm. Two additional deaths were reported after the 8/27/2020 cut-off date and both were from the placebo group. The all-cause deaths in placebo arm were 20 (20.4%) and in CD24Fc treatment arm were 12 (12.1%).

Serious Adverse Events: There were 46 serious adverse events (SAE) in 39 subjects recorded in the electronic data capture (EDC) system, none of which were related to the investigational drug. 25 SAE events from 18 subjects were from the CD24Fc treatment arm, and 21 SAE events from 21 subjects were from the placebo arm.

Clinical Improvement and Recovery: Among 197 randomized subjects, 146 subjects achieved clinical improvement and recovery from COVID-19 disease. 81 subjects were from the CD24Fc treatment arm; 65 subjects were from the placebo arm. The log rank HR=1.607 (95% CI: 1.158 to 2.231) and a P value of 0.005. The protocol prespecified efficacy boundary is P=0.0147. The interim analysis on the primary endpoint of clinical improvement and recovery exceeds the efficacy requirement in interim analysis of P<0.0147.

Sample size re-estimation: The conditional power at interim analysis (70% of 208 events) is >95% assuming the current observed data trend will continue for the remaining 30% events. This is based on the prespecified sample size re-estimation rule that if the interim conditional power is >85%, no sample size adjustment is necessary. For purposes of this section of the invention disclosure an “event” is when a patient achieves clinical improvement. The time to clinical improvement is defined as an improvement of patient's clinical diagnosis from baseline (score 3 or 4 or 2, NIAID ordinal scale) to 5 or above, if sustained without a drop to below 5 within 28 days.

Study Objectives:

Primary:

The primary objective of the Phase III study is to evaluate the safety and efficacy of adding CD24Fc to the common COVID-19 disease treatments by comparing COVID disease status improvement between patients that received CD24Fc versus a placebo within the 28 day period from randomization.

Secondary:

Secondary objectives are to compare, for each treatment arm, the proportion of patients who died or had respiratory failure (defined as the need for mechanical ventilation, extra corporal membrane oxygenation (ECMO), non-invasive ventilation, or high flow oxygen devices), the time to disease progression, the rate of all-cause death, the proportion of death or respiratory failure, rates of hospital discharge time, rate of duration of mechanical ventilation, duration of mechanical ventilation, use of pressors, rate of duration of extracorporeal membrane oxygenation, rate of duration of supplemental oxygen, the length of hospital stay, and the changes in absolute lymphocyte count and markers of inflammation.

Primary Endpoint:

Time to improvement in clinical status: the time (in days) to the improvement of clinical status from “scale 3 or 4” to “scale 5 or higher” and sustained without a drop to below scale 5, based on NIAID 8-point ordinal scales within 28 days from randomization.

Secondary Endpoints:

Proportion of patients who died or had respiratory failure, defined as the need for mechanical ventilation, ECMO, non-invasive ventilation, or high flow oxygen devices, at Day 29;

Time to disease progression in clinical status: the time (in days) for progression from scale 3 or 4 to scale 1 or 2, or 2 to 1, based on NIAID ordinal scale with 28 days from randomization;

All-cause mortality at Day 29;

Proportion of clinical relapse, as defined by rate of return to oxygen support for more than 1 day within 28 days from randomization after initial recovery;

Conversion rate of clinical status on day 8 (proportion of subjects who changed from “scale 3 or 4” to “scale 5 or higher” on NIAID ordinal scale);

Conversion rate of clinical status on day 15 (proportion of subjects who changed from “scale 3 or 4” to “scale 5 or higher” on NIAID ordinal scale);

The hospitalization discharge time;

Duration of mechanical ventilation (intermittent mandatory ventilation (IMV), non-invasive ventilation (MV) (days);

Duration of pressors;

Duration of extracorporeal membrane oxygenation (days);

Duration of oxygen therapy (oxygen inhalation by nasal cannula or mask) (days);

Length of hospital stay (days);

Absolute lymphocyte count;

D-dimer concentration in the plasma.

Study Population: Hospitalized severe and critical adult patients with COVID-19 disease (MAID scale 3 or 4).

The patient eligibility criteria are shown in Table 1.

TABLE 1
Inclusion criteria, exclusion criteria and NIAID 8-point scale system

| | | | | NIAID 8-point scale for | |
|---|---|---|---|---|---|
| Inclusion Criteria | | Exclusion Criteria | | COVID-19 | |
| 1. | Age ≥ 18 years | 1. | Patients with COVID 19 | 1. | Death; |
| 2. | Male or Female, | | in NIAID 8-point ordinal | 2. | Hospitalized, on |
| | Female pregnancy | | score 2 (Hospitalized, on | | invasive mechanical |
| | test negative | | invasive mechanical | | ventilation or |
| 3. | Diagnosed with | | ventilation or | | extracorporeal |
| | COVID-19 and | | extracorporeal membrane | | membrane oxygenation |
| | confirmed SARS- | | oxygenation (ECMO)). | | (ECMO); |
| | CoV-2 infection. | 2. | Patients with documented | 3. | Hospitalized, on non- |
| | prior positive viral | | bacterial/fungal | | invasive ventilation or |
| | results allowed, | | infections. | | high flow oxygen |
| 4. | Able to sign the | 3. | Patients with Sepsis or | | devices; |
| | consent form. | | septic shock | 4. | Hospitalized, requiring |
| 5. | Hospitalized and | 4. | Patients who are | | supplemental oxygen; |
| | requiring oxygen | | pregnant, breastfeeding, | 5. | Hospitalized, not |
| | support, NIAID 8- | | or have a positive | | requiring supplemental |
| | point ordinal score | | pregnancy test result | | oxygen - requiring |
| | 3 to 4, regardless | | before enrollment. | | ongoing medical care |
| | of ARDS. | 5. | Severe liver damage | | (COVID-19 related or |
| 6. | Women of | | (AST > 5 times the upper | | otherwise); |
| | childbearing | | limit). | 6. | Hospitalized, not |
| | potential, under | 6. | Patients with known | | requiring supplemental |
| | the age of 54 years, | | severe renal | | oxygen - no longer |
| | who use adequate | 7. | impairment (creatinine | | requires ongoing |
| | contraception and | | clearance ≤ 30 mL/min). | | medical care; |
| | who agree to use | | The investigator believes | 7. | Not hospitalized, |
| | adequate | | that participating in the | | limitation on activities |
| | contraception for | | trial is not in the best | | and/or requiring home |
| | the duration of the | | interests of the patient, or | | oxygen; |
| | study. | | the investigator considers | 8. | Not hospitalized, no |
| | | | unsuitable for enrollment | | limitations on |
| | | | (such as unpredictable | | activities. |
| | | | risks or subject | | |
| | | | compliance issues). | | |

Treatment Description:

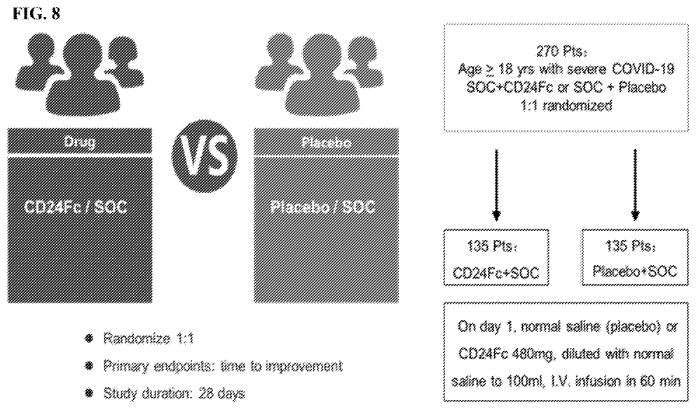

The Phase III study was a randomized double-blind placebo-controlled study in 270 subjects (FIG. 8). Patients were randomized 1:1 to receive one of the following treatments as single dose administration on Day 1.

Arm A: CD24Fc, 480 mg, diluted to 100 ml with normal saline, IV infusion in 60 minutes.

Arm B: placebo, normal saline 100 ml, IV infusion in 60 minutes.

The best available treatment and supportive care were given to all subjects according to local institutional guideline. Those subjects who use immune modulators, such as IL-6/IL-6R antagonists, or experimental antiviral drugs, such as remdesivir, are allowed to participate in the trial.

Accrual Objective: The study will enroll 270 patients, or 135 per arm.

Accrual Period: The estimated accrual period is 5 months.

Study Duration: Patients are followed for 28 days.

Interim Analysis Purpose

The study includes an interim efficacy analysis at 70% events, which is 146 events, for the purpose of sample re-estimation and whether the efficacy data suggest early termination either for safety concern or for efficacy based on the threshold listed in the DSMB charter. The prespecified efficacy boundary is P=0.0147. Chen's method is used to re-estimate the sample size. The final primary analysis will be carried out after all 270 subjects have completed the study.

Analysis Population

The following analysis population is used to analyze the results from the analyses.

Intended-To-Treat Set (ITT)

Defined as all subjects who are randomized to participate in the trial. The ITT population is the main analysis population for the efficacy analysis of this study. Subjects are analyzed based on the treatment group in which they are randomized.

Per Protocol Set (PPS)

Per Protocol Set is a subset of the ITT population. It refers to all subjects who have received protocol-specified single-dose treatment without significant protocol deviations that significantly affect the main efficacy. PPS-based analysis complements ITT-based analysis as a supporting analysis. Subjects are analyzed based on the treatment arm they are randomized.

Safety Data Set (SS)

Safety Data Set is defined as all subjects who received at least one test drug. The safety analysis population is the main analysis population for safety data. Subjects are analyzed based on their actual assigned treatment arm.

Study Endpoints

The primary efficacy endpoint and SAE are evaluated.

Primary Endpoints: time to improvement in clinical status: the time (in days) required for improvement from scale 3-4 to scale ≥5 and sustained without a drop to below scale 5 based on MAID ordinal scales (Table 1) within 28 days from randomization.

Safety Evaluation: Severe Adverse events including mortalities.

Subject Disposition

Descriptive statistics by treatment are used to summarize the number of subjects screened, the number of screening failures, enrollment, randomization, treatment, entry into each analysis set (and reasons for excluding each analysis set), withdrawal from the study, and follow-up. The descriptive statistics that include numbers and percentages of subjects who entered each analysis set and withdrew from the study are summarized by treatment arms and study sites.

Enrollment Overview

Number of Subjects (as of Aug. 27, 2020):

Enrolled: 203 subjects.

Screen failure: 6 subjects.

Randomized: 197 subjects.

Randomized but not dosed: 3 subjects.

Completed treatment: 194 subjects.

Completed study with 28 days follow up: 141 subjects.

Discontinued due to death: 26 subjects.

In study: 30 subjects.

Demographics, and Baseline Characteristics

Demographic information and baseline characteristics for all randomized subjects are summarized using descriptive statistical methods based on treatment arm. The following table presents the basic demographic information of 197 patients.

TABLE 2
Demographics and baseline NIAID scores

| | Arm A | | Arm B | | | |
|---|---|---|---|---|---|---|
| | CD24Fc 480 mg | | Placebo | | Overall | |
| | (N = 99) | | (N = 98) | | (N = 197) | |
| | n (%) | | n (%) | | n (%) | |
| Age (Years) | | | | | | |
| N | 96 | | 97 | | 193 | |
| Mean (SD) | 58.3 | (14.01) | 56.9 | (14.85) | 57.6 | (14.42) |
| Median | 58.0 | | 59.0 | | 59.0 | |
| Q1, Q3 | 48.0, 69.5 | | 46.0, 67.0 | | 47.0, 68.0 | |
| Min, Max | 27, 86 | | 23, 91 | | 23, 91 | |
| Gender, n (%) | | | | | | |
| Male | 56 | (56.6) | 61 | (62.2) | 117 | (59.4) |
| Female | 40 | (40.4) | 36 | (36.7) | 76 | (38.6) |
| Missing | 3 | (3.0) | 1 | (1.0) | 4 | (2.0) |
| Race, n (%) | | | | | | |
| White | 31 | (31.3) | 32 | (32.7) | 63 | (32.0) |
| Black or African American | 25 | (25.3) | 18 | (18.4) | 43 | (21.8) |
| Asian | 2 | (2.0) | 1 | (1.0) | 3 | (1.5) |
| Others | 38 | (38.4) | 46 | (46.9) | 84 | (42.6) |
| Missing | 3 | (3.0) | 1 | (1.0) | 4 | (2.0) |
| Ethnicity, n (%) | | | | | | |
| Hispanic or Latino | 36 | (36.4) | 43 | (43.9) | 79 | (40.1) |
| Not Hispanic or Latino | 62 | (62.6) | 55 | (56.1) | 117 | (59.4) |
| Missing | 1 | (1.0) | 0 | | 1 | (0.5) |
| Median Duration of Symptom | 10.0 | (7.0, 13.0) | 10.0 | (6.0, 12.0) | 10.0 | (6.5, 12.0) |
| Onset to Randomization - Days | | | | | | |
| (Q1-Q3) | | | | | | |
| NIAID Rating at Baseline | | | | | | |
| 3: Non-invasive MV or High Flow | 48 | (48.5) | 56 | (57.1) | 104 | (52.8) |
| Oxygen Devices | | | | | | |
| 4: Supplemental Oxygen | 51 | (51.5) | 42 | (42.9) | 93 | (47.2) |

Efficacy Analysis

Primary Efficacy Analysis, Time to Clinical Improvement (TTCI)

Clinical improvement is defined as an improvement of patient's clinical diagnosis from baseline (scale 3 or 4, NIAID ordinal score) to 5 or above, if sustained without a drop to below 5 within the 28 days from randomization. Hospital discharge date (MAID scale 7 or 8) is an important time point. The date on which the clinical status evaluation is ≥5 or the date of hospital discharge, whichever comes first, is used as the TTCI date.

The record of re-hospitalization with oxygen therapy is required to modify the TTCI date. The new TTCI date should be the time from randomization to the latest clinical status evaluation of ≥5 or the date of the latest hospital discharge, whichever comes first.

Time to clinical improvement (days) is calculated as:

Time to Improvement=Date of(First Clinical Improvement/hospital discharge)−Date of Randomization

The hospital discharge to home due to clinical improvement is scored as NIAID scale ≥7. The date that clinical status evaluation ≥5 or the date of hospital discharge, whichever comes first, is used as the clinical improvement date. The number of patients that achieve clinical improvement within 28 days from randomization, time to clinical improvement, and cumulative clinical improvement rate at Day 3, 6, 9, 12, 15, 18, 21 and 28 are summarized by the treatment arm. Time to clinical improvement is listed by subject.

Subjects who are not lost to follow-up or do not experience improvement are censored on the actual date of the Day 29 visit. Subjects without improvement but lost to follow-up are censored on the last date of follow-up. Subjects with death within the 28 days are censored on D29. Subjects without improvement but withdrawal from the study are censored at the withdrawal date.

The Log-Rank Test is used to compare the following two sets of curves to test the following hypotheses:

H0:St(t)=Sp(t)vs.H1:St(t)>Sp(t) for improvement;

Where St(t) represents the improvement probability function of the treatment group, and Sp(t) represents the improvement probability function of the control group.

The probability function of clinical improvement is estimated by Kaplan-Meier method. The at-risk numbers from each group at Day 0 is the ITT on each group. The median time and its 95% confidence interval for each group is reported. The 95% confidence interval for the median time is estimated using the Brookmeyer-Crowley method with log-log function conversion to achieve a normal approximation. The hazard ratio is estimated by the Cox proportional hazard model. Cumulative improvement rates estimated by the KM method for Day 3, 6, 9, 12, 15, 18, 21 and 28, and their 95% confidence intervals is also reported. All analyses above are repeated for subjects with NIAID score=3 at baseline and subjects with MAID score=4 at baseline. All analyses are repeated for gender, race and ethnicity subgroups.

Data are fitted to the following models using different co-variates separately: treatment+baseline NIAID score, treatment+gender, treatment+race, and treatment+ethnicity.

Primary Efficacy Unblinded Results

Overall

From the available data on 197 patients, there were 146 subjects with clinical improvements and recoveries. For the 146 events occurred within the 28 days since randomization, the median time of TTCI is 7 days (95% CI: 6.00 to 10.00) overall. The 28-day recovery rate is overall 74% estimated for by K-M method.

Among the 146 events, 81 events are from the CD24Fc treatment group of 99 (Table 3). The median time of TTCI is 6 days (95% CI: 5.00 to 8.00). The 28-day recovery rate is 81.8% estimated by K-M method.

There are 65 events from the placebo group of 98 (Table 4). The median time of TTCI is 10 days (95% CI: 7.00 to 15.00). The 28-day recovery rate is 66.3% estimated for by K-M method.

TABLE 3
Raw data on 81 TTCI events (CD24Fc arm)

| TTCI, Days | 1-3 | 4-6 | 7-9 | 10-12 | 13-15 | 16-18 | 19-21 | 22-28 | >28 | Total |
|---|---|---|---|---|---|---|---|---|---|---|
| N | 29 | 22 | 14 | 8 | 4 | 0 | 0 | 4 | 0 | 81 |
| Cum. No. | 29 | 51 | 65 | 73 | 77 | 77 | 77 | 81 | 81 | |
| Days | 3 | 6 | 9 | 12 | 15 | 18 | 21 | 28 | | |
| Cumulative | 29% | 52% | 66% | 74% | 78% | 78% | 78% | 82% | N/A | N/A |
| Rate (%) | | | | | | | | | | |

TABLE 4
Raw data on 65 TTCI events (Placebo arm)

| TTCI, Days | 1-3 | 4-6 | 7-9 | 10-12 | 13-15 | 16-18 | 19-21 | 22-28 | >28 | Total |
|---|---|---|---|---|---|---|---|---|---|---|
| N | 17 | 17 | 11 | 5 | 8 | 3 | 1 | 3 | 0 | 65 |
| Cum. No. | 17 | 34 | 45 | 50 | 58 | 61 | 62 | 65 | 65 | |
| Days | 3 | 6 | 9 | 12 | 15 | 18 | 21 | 28 | | |
| Cumulative | 17% | 35% | 46% | 51% | 59% | 62% | 63% | 66% | N/A | N/A |
| Rate (%) | | | | | | | | | | |

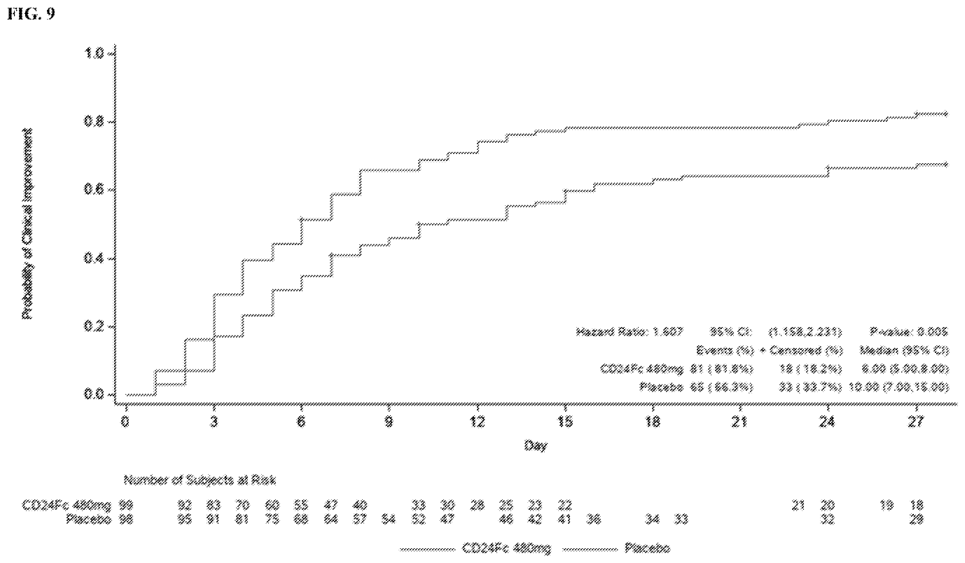

A K-M method was used to evaluate the CD24Fc treatment effects in severe and critical COVID-19 patients (FIG. 9). The hazard ratio is 1.607 (95% CI: 1.158 to 2.231). P=0.005. Based on the interim analysis of the observed 146 events, we performed conditional power calculation.

Assuming the additional 30% TTCI events (˜63) follows the same data trend (the hazard ratio=1.607) as observed in the first 70% (146) events, the conditional power is >95%.

The conditional power was evaluated using statistical software East® 6.4, Cytel, Waltham, Mass.

FIG. 9 shows Kaplan-Meier estimates of cumulative time to clinical improvement. Cumulative recovery estimates are shown in the overall population.

Subgroup: Baseline MAID Score

Among the 197 cases, 93 (47.2%) patients had baseline MAID score of 4, which corresponds to being hospitalized with supplemental oxygen support. These patients required nasal cannula to provide supplemental oxygen in the volume of 2 L/min to 6 L/min. There were 84 events occurred within the 28 days from randomization. 50 events were from the CD24Fc treatment arm (N=51). The median time of TTCI for MAID score 4 in the CD24Fc arm is 4 days (95% CI: 3 to 5). The 28-day recovery rate is 98% estimated by K-M method (Table 5).

TABLE 5
Raw data on 50 TTCI events in baseline NIAID score 4 in CD24Fc arm

| TTCI, Days | 1-3 | 4-6 | 7-9 | 10-12 | 13-15 | 16-18 | 19-21 | 22-28 | Total |
|---|---|---|---|---|---|---|---|---|---|
| N | 25 | 12 | 10 | 1 | 1 | 0 | 0 | 1 | 50 |
| Cum. No. | 25 | 37 | 47 | 48 | 49 | 49 | 49 | 50 | |
| Day | 3 | 6 | 9 | 12 | 15 | 18 | 21 | 28 | |
| Cumulative Rate (%) | 49% | 73% | 92% | 94% | 96% | 96% | 96% | 98% | N/A |

There were 84 events that occurred within the 28 days from randomization. 34 events were from the placebo arm (N=42). The median time of TTCI for MAID score 4 in the placebo arm is 5.5 days (95% CI: 4 to 8). The 28-day recovery rate is 81% estimated by K-M method (Table 6).

TABLE 6
Raw data on 34 TTCI events in baseline NIAID score 4 in Placebo arm

| TTCI, Days | 1-3 | 4-6 | 7-9 | 10-12 | 13-15 | 16-18 | 19-21 | 22-28 | Total |
|---|---|---|---|---|---|---|---|---|---|
| N | 13 | 10 | 5 | 2 | 1 | 0 | 1 | 2 | 34 |
| Cum. No. | 13 | 23 | 28 | 30 | 31 | 31 | 32 | 34 | |
| Day | 3 | 6 | 9 | 12 | 15 | 18 | 21 | 28 | |
| Cumulative Rate (%) | 31% | 55% | 67% | 71% | 74% | 74% | 76% | 81% | N/A |

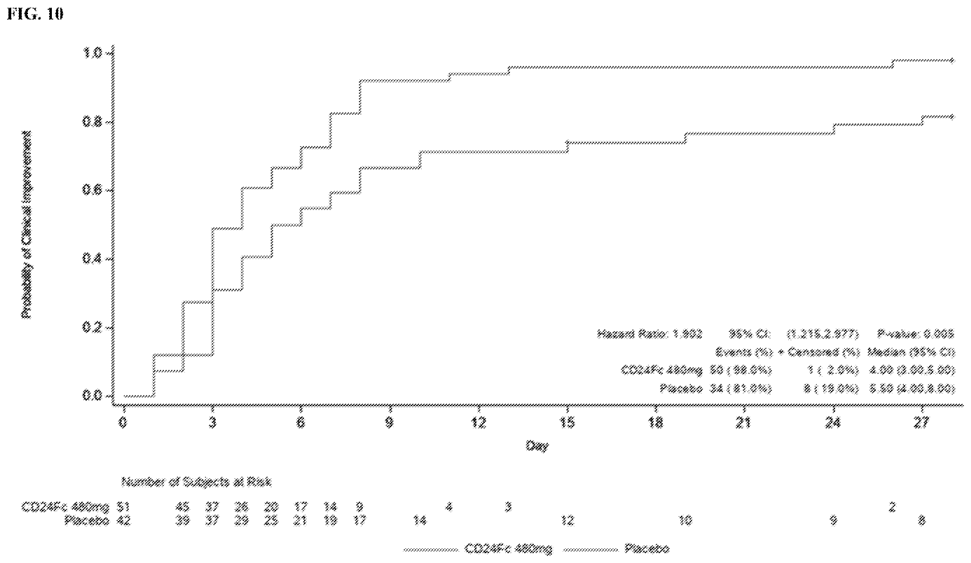

A K-M method was used to evaluate the CD24Fc treatment effects in severe COVID-19 patients with MAID score 4 (FIG. 10). The hazard ratio was 1.902 (95% CI: 1.215 to 2.977). P=0.005.

Among the 197 cases, 104 (52.8%) patients had baseline MAID score of 3, which is corresponds to hospitalization with non-invasive mechanical ventilation or high flow oxygen devices. These patients required an oxygen mask, several different types of non-invasive ventilation instrument or high flow oxygen device to provide pure oxygen in the volume of 8 L/min to 40 L/min. There were 62 events that occurred within the 28 days from randomization. 31 events were from the CD24Fc treatment arm (N=48). The median time of TTCI for MAID score 3 is 12 days (95% CI: 8 to 24). The 28 day recovery rate is 64.6% estimated by K-M method (Table 7).

TABLE 7
Raw data on 31 TTCI events in baseline NIAID score 3 in CD24Fc arm

| TTCI, Days | 1-3 | 4-6 | 7-9 | 10-12 | 13-15 | 16-18 | 19-21 | 22-28 | Total |
|---|---|---|---|---|---|---|---|---|---|
| N | 4 | 10 | 4 | 7 | 3 | 0 | 0 | 3 | 31 |
| Cum. No. | 4 | 14 | 18 | 25 | 28 | 28 | 28 | 31 | |
| Day | 3 | 6 | 9 | 12 | 15 | 18 | 21 | 28 | |
| Cumulative Rate (%) | 8% | 29% | 38% | 52% | 58% | 58% | 58% | 65% | N/A |

For the 62 events occurred within the 28 days from randomization, 31 events were from the placebo arm (N=56). The median time of TTCI for MAID score 3 is 16 days (95% CI: NC to NC). The 28 day recovery rate is 55.4% estimated by K-M method (Table 8).

TABLE 8
Raw data on 31 TTCI events in baseline NIAID score 3 in placebo arm

| | | | | | | | | | |
|---|---|---|---|---|---|---|---|---|---|
| N | 4 | 7 | 6 | 3 | 7 | 3 | 0 | 1 | 31 |
| Cum. No. | 4 | 11 | 17 | 20 | 27 | 30 | 30 | 31 | |
| Day | 3 | 6 | 9 | 12 | 15 | 18 | 21 | 28 | |
| Cumulative Rate (%) | 7% | 20% | 30% | 36% | 48% | 54% | 54% | 55% | N/A |

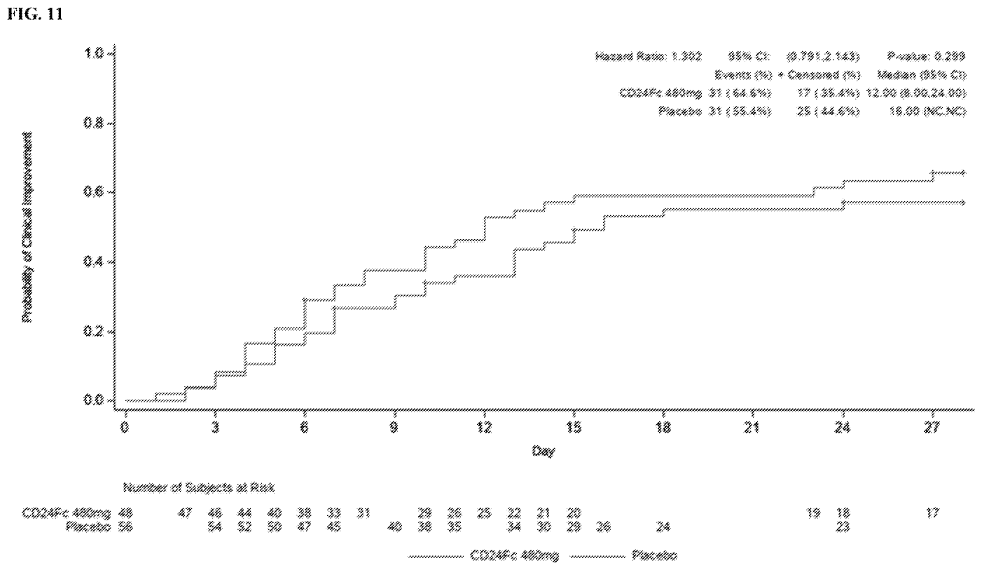

A K-M method was used to evaluate the CD24Fc treatment effects in critical COVID-19 patients with MAID score 3 (with non-invasive ventilation or high flow oxygen devices) (FIG. 11). The hazard ratio is 1.302 (95% CI: 0.791 to 2.143). P=0.299.

Subgroup: Gender

Among the 197 cases, 117 (59.4%) patients are male.

There were 89 events that occurred within the 28 days from randomization, 48 events were from the CD24Fc treatment arm. The median time of TTCI for males is 6.50 days (95% CI: 4.00 to 8.00) in the CD24Fc treatment arm and the 28-day recovery rate is 85.7% estimated by KM method.

For the 89 events that occurred within the 28 days from randomization, 41 events were from the placebo arm. The median time of TTCI for males is 13.00 days (95% CI: 7.00 to 19.00) in the placebo arm. The 28-day recovery rate is 67.2% estimated by K-M method.

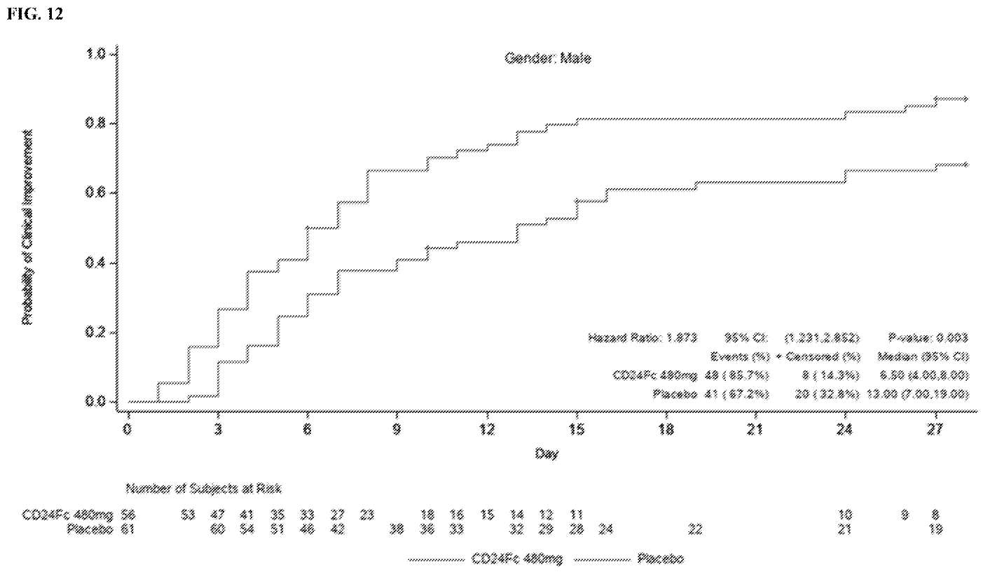

A K-M method was used to evaluate the CD24Fc treatment effects in male COVID-19 patients (FIG. 12). The hazard ratio is 1.873 (95% CI: 1.231 to 2.852). P=0.003.

Among the 197 cases, 76 (38.6%) patients are female.

There were 53 events that occurred within the 28 days from randomization; 30 events were from the CD24Fc treatment arm. The median time of TTCI for females is 6.00 days (95% CI: 4.00 to 10.00) in the CD24Fc treatment arm and the 28-day recovery rate is 75.0% estimated by KM method.

For the 53 events that occurred within the 28 days from randomization, 23 events were from the placebo arm. The median time of TTCI for females is 8.00 days (95% CI: NC to NC) in the placebo arm. The 28-day recovery rate is 63.9% estimated by K-M method.

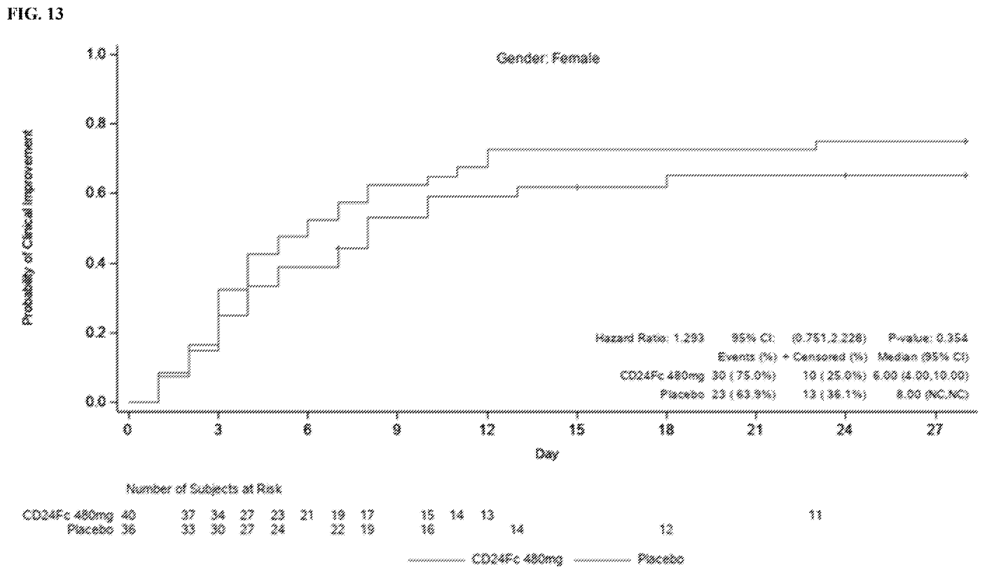

We used K-M method to evaluate the CD24Fc treatment effects in female COVID-19 patients (FIG. 13). The hazard ratio is 1.293 (95% CI: 0.751 to 2.228). P=0.354.

Subgroup: Race

Among the 197 cases, 63 (32.0%) patients are White.

There were 41 events that occurred within 28 days from randomization, 25 events were from the CD24Fc treatment arm. The median time of TTCI for White patients is 6.00 days (95% CI: 4.00 to 8.00) in the CD24Fc treatment arm, and the 28-day recovery rate is 80.6% estimated by KM method.

For the 41 events that occurred within 28 days from randomization, 16 events were from the placebo arm. The median time of TTCI for White patients is 16.00 days (95% CI: NC to NC) in the placebo arm. The 28-day recovery rate is 50.0% estimated by K-M method.

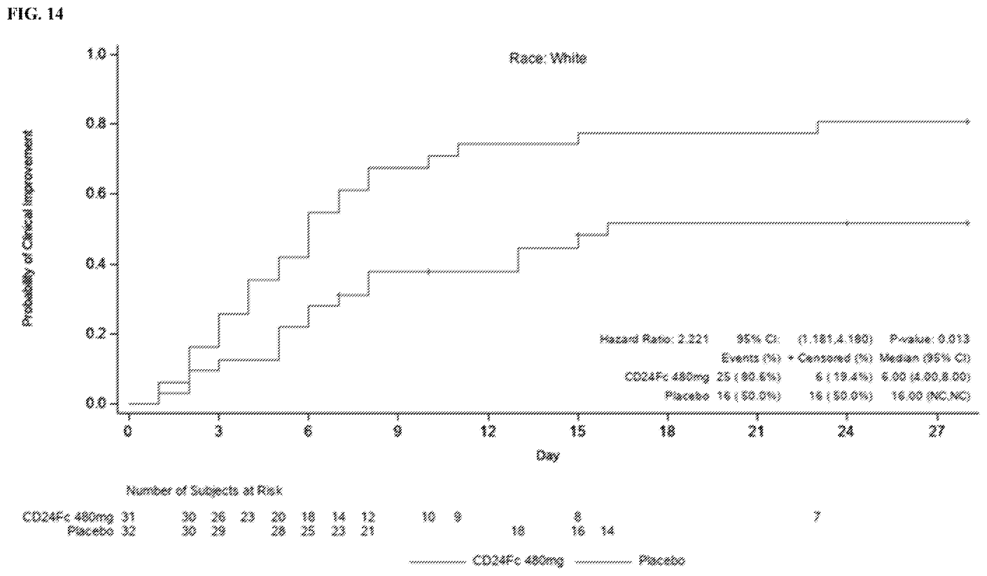

A K-M was method to evaluate the CD24Fc treatment effects in White COVID-19 patients (FIG. 14). The hazard ratio is 2.221 (95% CI: 1.181 to 4.180). P=0.013.

Among the 197 cases, 43 (21.8%) patients are Black or African American.

There were 30 events the occurred within 28 days from randomization, 19 events were from the CD24Fc treatment arm. The median time of TTCI for Black or African American patients is 5.00 days (95% CI: 3.00 to 10.00) in the CD24Fc treatment arm and the 28-day recovery rate is 76.0% estimated by KM method.

For the 30 events the occurred within 28 days from randomization, 11 events were from the placebo arm. The median time of TTCI for Black or African American patients is 11.50 days (95% CI: NC to NC) in the placebo arm. The 28-day recovery rate is 61.1% estimated by K-M method.

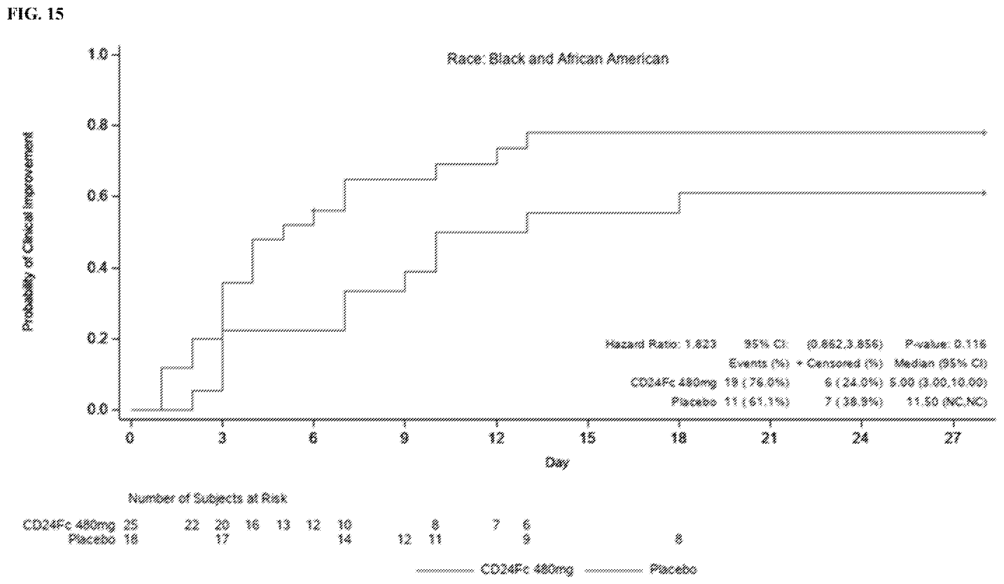

We used K-M method to evaluate the CD24Fc treatment effects in Black or African American COVID-19 patients (FIG. 15). The hazard ratio is 1.823 (95% CI: 0.862 to 3.856). P=0.116.

Among the 197 cases, 87 (44.2%) patients are other races.

There were 71 events the occurred within 28 days from randomization, 34 events were from the CD24Fc treatment arm. The median time of TTCI for other races patients is 7.50 days (95% CI: 4.00 to 10.00) in the CD24Fc treatment arm and the 28-day recovery rate is 85.0% estimated by KM method.

For the 71 events the occurred within 28 days from randomization, 37 events were from the placebo arm. The median time of TTCI for other races patients is 8.00 days (95% CI: 5.00 to 14.00) in the placebo arm. The 28-day recovery rate is 78.7% estimated by K-M method.

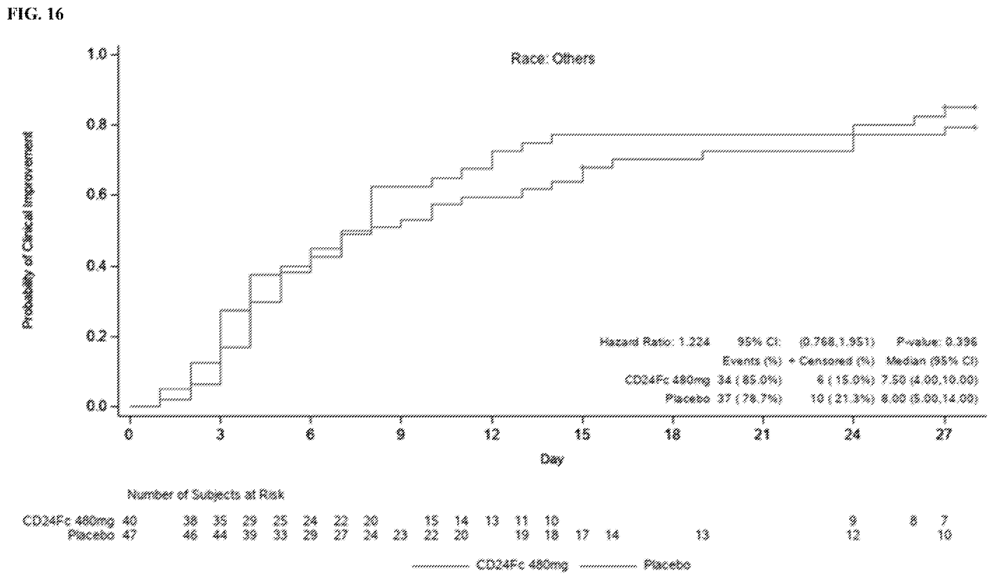

We used K-M method to evaluate the CD24Fc treatment effects in other races COVID-19 patients (FIG. 16). The hazard ratio is 1.224 (95% CI: 0.768 to 1.951). P=0.396.

Subgroup: Ethnicity

Among the 197 cases, 79 (40.1%) patients are Hispanic or Latino.

There were 63 events the occurred within 28 days from randomization, 30 events were from the CD24Fc treatment arm. The median time of TTCI for Hispanic or Latino patients is 7.50 days (95% CI: 4.00 to 11.00) in the CD24Fc treatment arm and the 28-day recovery rate is 83.3% estimated by KM method.

For the 63 events that occurred within 28 days from randomization, 33 events were from the placebo arm. The median time of TTCI for Hispanic or Latino patients is 9.00 days (95% CI: 5.00 to 15.00) in the placebo arm. The 28-day recovery rate is 76.7% estimated by K-M method.

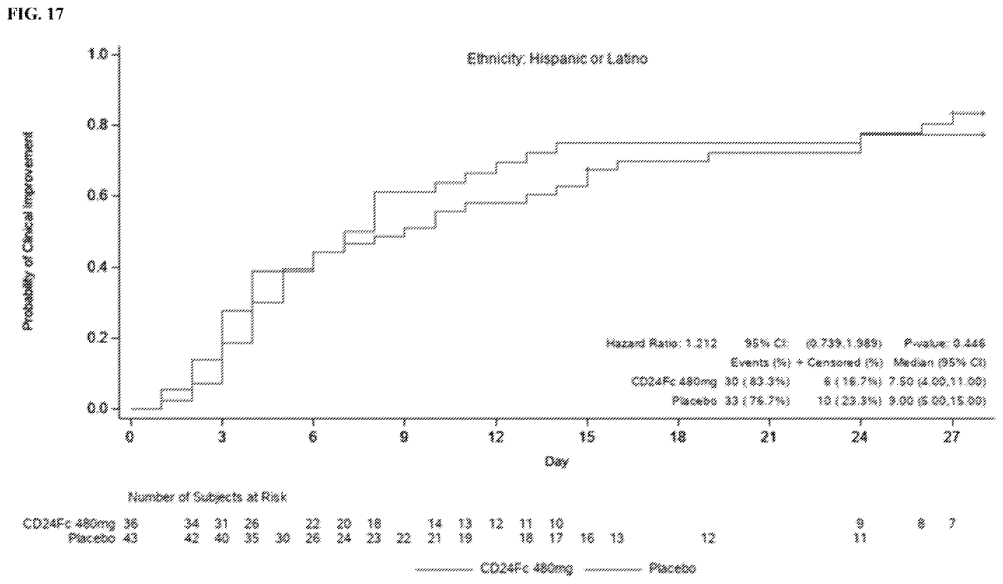

A K-M method was to evaluate the CD24Fc treatment effects in Hispanic or Latino COVID-19 patients (FIG. 17). The hazard ratio is 1.212 (95% CI: 0.739 to 1.989). P=0.446.

Among the 197 cases, 117 (59.4%) patients are not Hispanic or Latino.

There were 82 events that occurred within 28 days from randomization, 50 events were from the CD24Fc treatment arm. The median time of TTCI for patients not Hispanic or Latino is 6.00 days (95% CI: 4.00 to 7.00) in the CD24Fc treatment arm and the 28-day recovery rate is 80.6% estimated by KM method.

For the 82 events that occurred within 28 days from randomization, 32 events were from the placebo arm. The median time of TTCI for patients not Hispanic or Latino is 13.00 days (95% CI: NC to NC) in the placebo arm. The 28-day recovery rate is 58.2% estimated by K-M method.

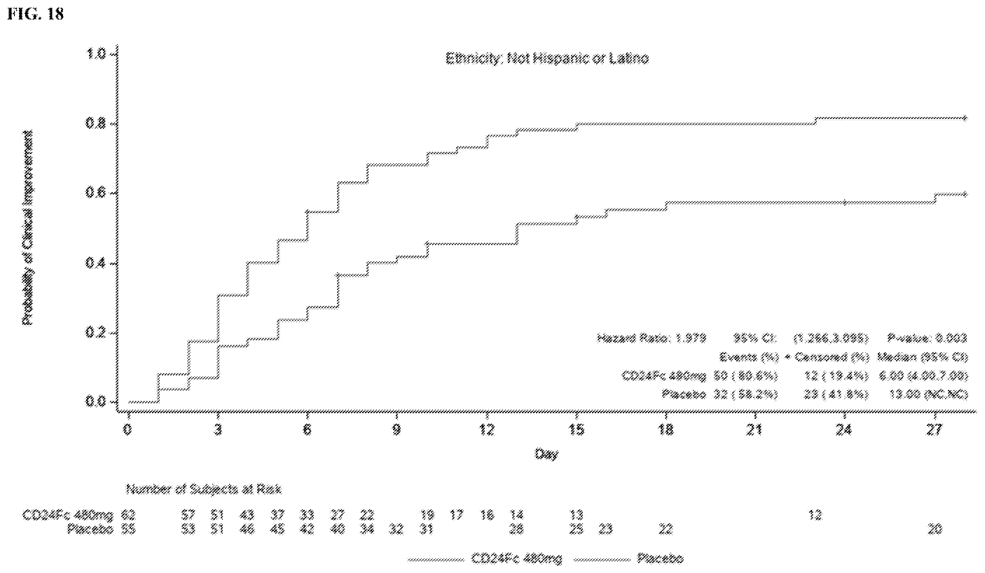

We used K-M method to evaluate the CD24Fc treatment effects in not Hispanic or Latino COVID-19 patients (FIG. 18). The hazard ratio is 1.979 (95% CI: 1.266 to 3.095). P=0.003.

Subgroup: Remdesivir as Part of Standard of Care

Among the 197 cases, 109 (55.3%) patients were given remdesivir as part of standard of care.

There were 44 patients in the CD24Fc treatment arm with remdesivir treatment. 35 patients were recovered. The median time of TTCI for remdesivir+CD24Fc treatment is 6.00 days (95% CI: 4.00 to 8.00) and the 28-day recovery rate is 81.0% estimated by K-M method.

There were 65 patients in the placebo arm with remdesivir treatment. 40 patients recovered. The median time of TTCI for remdesivir+placebo is 13.00 days (95% CI: 9.00 to 27.00) and the 28-day recovery rate is 62.8% estimated by K-M method.

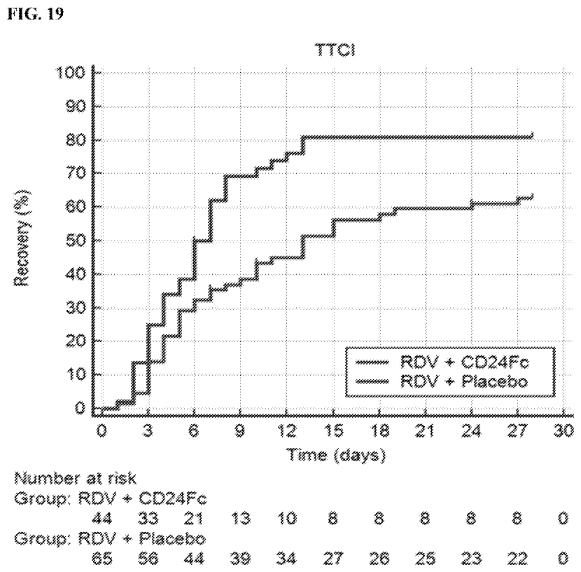

We used K-M method to evaluate the CD24Fc treatment effects in combination of remdesivir in comparison with remdesivir in severe and critical COVID-19 patients (FIG. 19). The hazard ratio is 1.997 (95% CI: 1.204 to 3.311). P=0.007.

Subgroup: Corticosteroids as Part of Standard of Care

Among the 197 cases, 97 (49.2%) patients were given corticosteroid, including dexamethasone, prednisone, methyl prednisolone, as part of standard of care.

There were 48 patients in the CD24Fc treatment arm with corticosteroid treatment. 38 patients were recovered. The median time of TTCI for corticosteroid+CD24Fc treatment is 5.00 days (95% CI: 3.00 to 8.00) and the 28-day recovery rate is 79.2% estimated by K-M method.

There were 49 patients in the placebo arm with corticosteroid treatment. 25 patients recovered. The median time of TTCI for corticosteroid+placebo is 27.00 days (95% CI: 7.00 to 27.00) and the 28-day recovery rate is 52.0% estimated by K-M method.

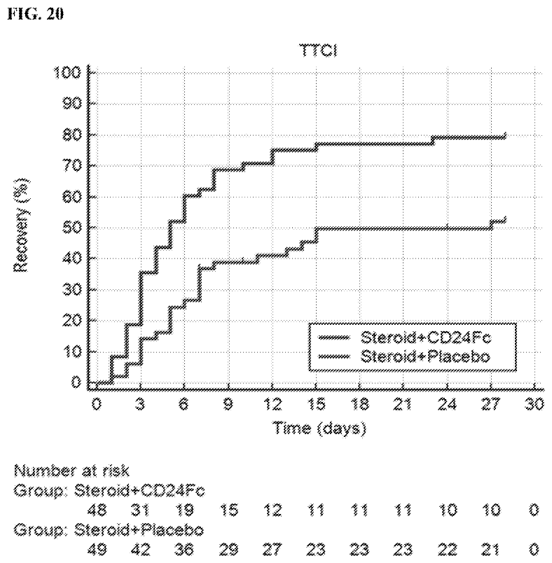

We used K-M method to evaluate the CD24Fc treatment effects in combination of corticosteroid in comparison with corticosteroid in severe and critical COVID-19 patients (FIG. 20). The hazard ratio is 2.427 (95% CI: 1.434 to 4.108). P=0.001.

Covariate: Baseline MAID Score

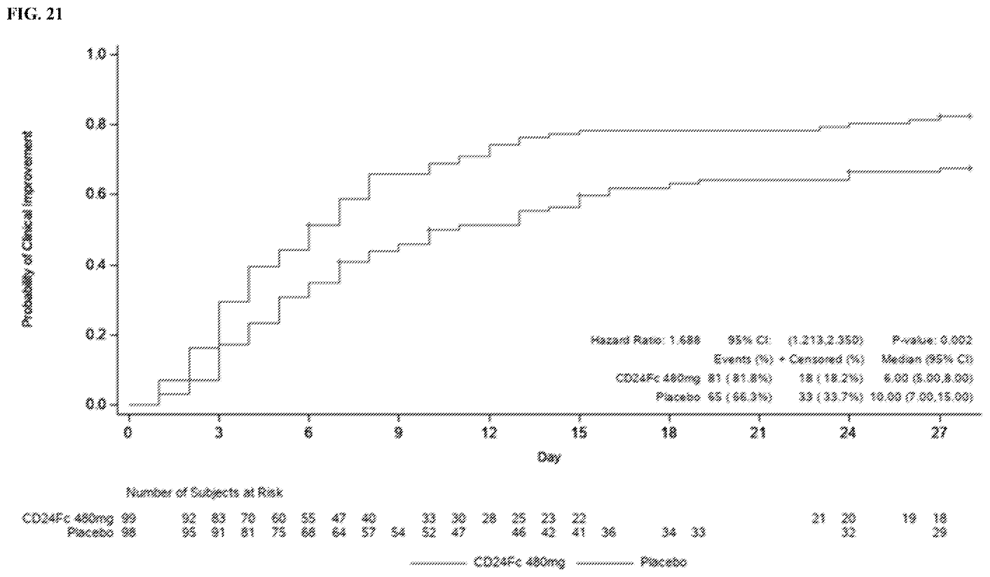

We used K-M method to evaluate the CD24Fc treatment effects in severe COVID-19 patients, using baseline MAID score as co-variate (FIG. 21). The hazard ratio is 1.688 (95% CI: 1.213 to 2.350). P=0.002.

Covariate: Gender

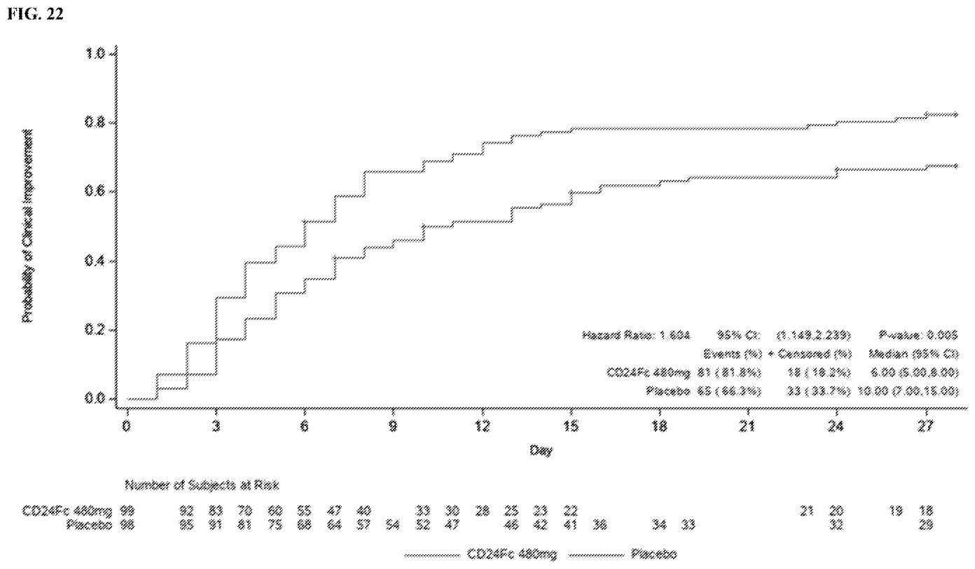

We used K-M method to evaluate the CD24Fc treatment effects in severe COVID-19 patients, using gender as co-variate (FIG. 22). The hazard ratio is 1.604 (95% CI: 1.149 to 2.239). P=0.005.

Co-variate: Race

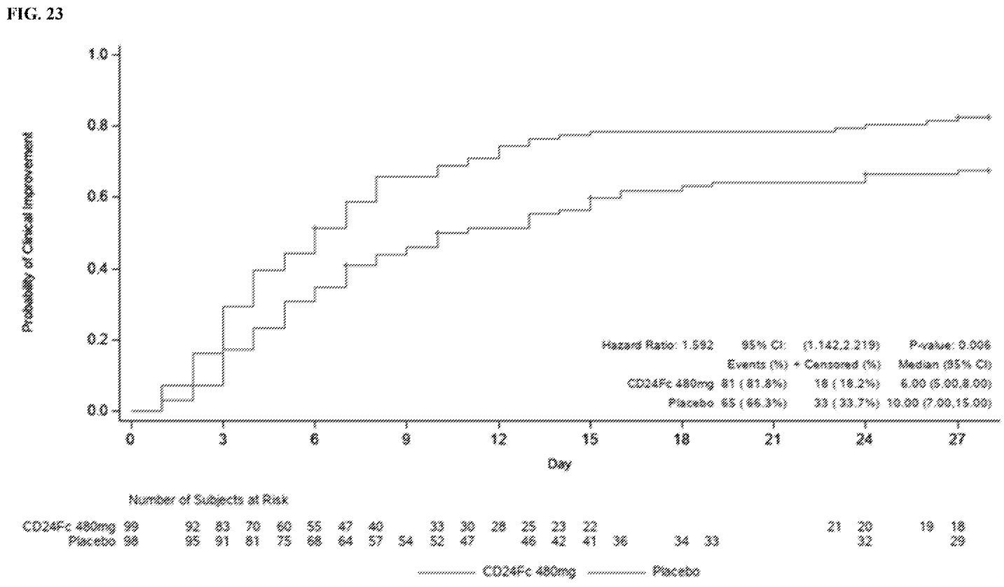

We used K-M method to evaluate the CD24Fc treatment effects in severe COVID-19 patients, using race as co-variate (FIG. 23). The hazard ratio is 1.592 (95% CI: 1.142 to 2.219). P=0.006.

Co-variate: Ethnicity

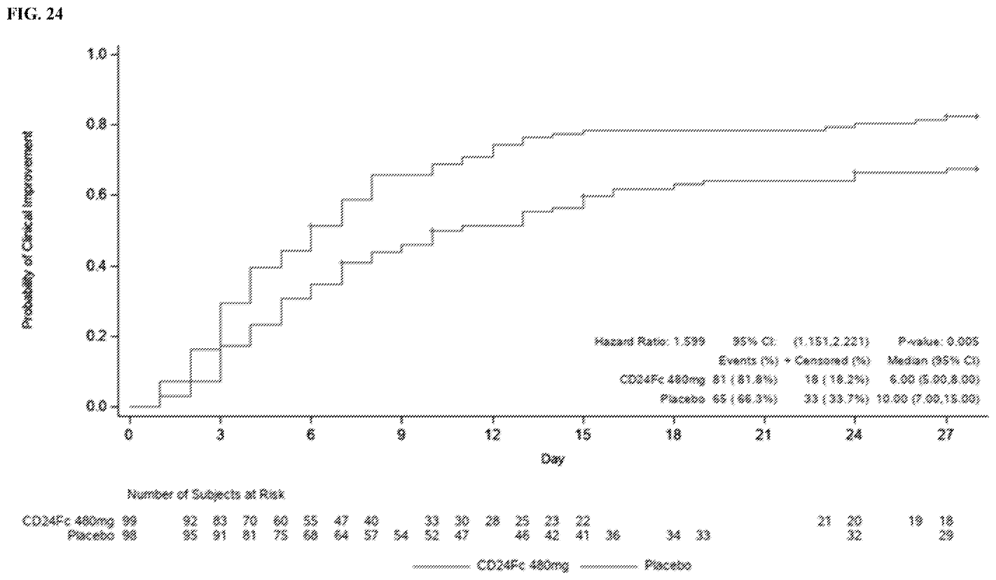

We used K-M method to evaluate the CD24Fc treatment effects in severe COVID-19 patients, using ethnicity as covariate (FIG. 24). The hazard ratio is 1.599 (95% CI: 1.151 to 2.221). P=0.005.

Efficacy analyses on secondary endpoints

Proportion of Death or Respiratory Failure at Day 29

Respiratory failure is defined as the need for any one of the following: 1) mechanical ventilation, 2) ECMO, 3) non-invasive ventilation, or 4) high flow oxygen devices. Proportion of death or respiratory failure at Day 29 are calculated as:

Proportion of Death at Day 29=Cumulative Number of Patients Died at Day 29/Total Number of Patients in ITT Population

Proportion of Respiratory Failure at Day 29=Cumulative Respiratory Failure Patients at Day 29/Total Number of Patients in ITT Population

Proportion of Death and Respiratory Failure at Day 29 were summarized by the treatment arm, including p-value of Chi-square test to compare the difference between the two treatment arms. All patients who were in respiratory failure at Day 29 or expired were listed.

TABLE 9
Combined death/RF by day 29, Chi-Squared, p-value

| | Arm A | Arm B | |
|---|---|---|---|
| | CD24Fc 480 mg | Placebo | |
| Death/Respiratory Failure | (N = 99) | (N = 98) | p-value [2] |
| N (%) | 15 (15.2) | 26 (26.5) | |
| 95% CI [1] | 8.7, 23.8 | 18.1, 36.4 | 0.0492 |

[1] 95% CI is obtained using the Clopper-Pearson method.
[2] P-value is based on Chi-Square Test.

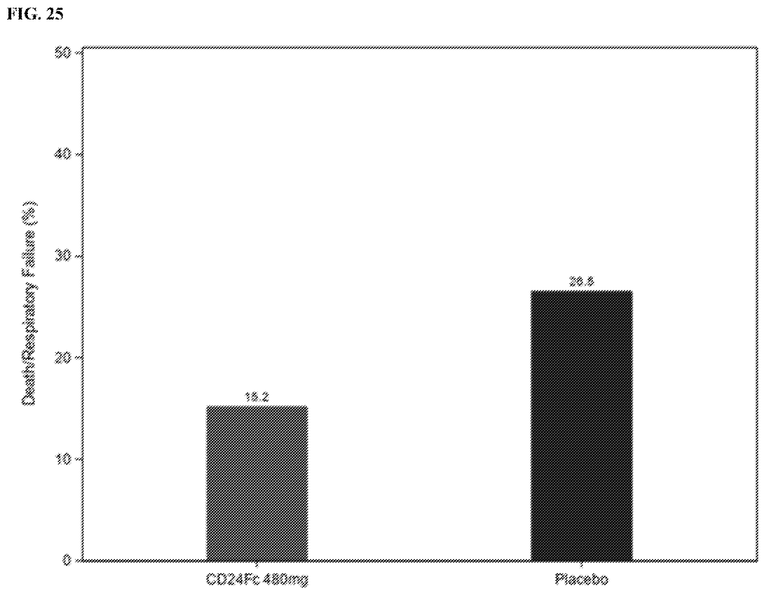

FIG. 25 shows death/RF on Day 29 by treatment assignment.

Among the 197 subjects with known information, 26 patients expired before 28 days, and 15 patients were in respiratory failure, defined as requiring: 1) mechanical ventilation, 2) ECMO, 3) non-invasive ventilation, or 4) high flow oxygen devices.

Among the 197 patients, 99 were in the CD24Fc treatment arm. In the CD24Fc arm, there were 11 deaths before 28 days and 4 patients in respiratory failure at Day 29. The total combined death/RF is 15 with the rate of 15.2%.

There were 98 subjects in the placebo arm. In the placebo arm, there were 15 deaths before the 28 days from randomization and 11 patients were in respiratory failure at Day 29. The total combined death/RF was 26 with the rate of 26.5%.

The Chi-square test p-value comparing combined death/RF between the two arms was 0.0492.

Disease Progression

Disease progression is defined as a progression from scale 3 or 4 to scale 2 or 1 during the observation period. Time to disease progression (days) is calculated as:

Time to Disease Progression=Date of First Disease Progression−Randomization Date

Subjects who withdrew from study were censored on the actual withdrawal date. Subjects lost to follow-up were censored on the last date of follow-up. The telemedicine record date was a follow up visit date.

Time to disease progression was listed by subject.

The probability function of disease progression was estimated by Kaplan-Meier method. The median time and its 95% confidence interval for each group was reported. The 95% confidence interval for the median time was estimated using the Brookmeyer-Crowley method with log-log function conversion to achieve a normal approximation. The progression rate was estimated by the layered Cox-Model. Cumulative progression rates estimated by the KM method for day 29 and the 95% confidence intervals were reported.

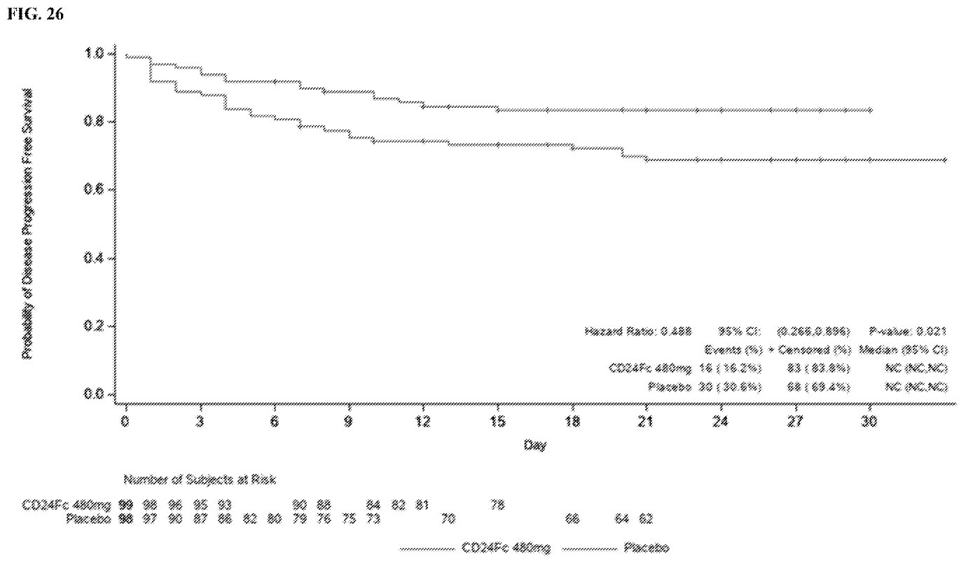

Among 197 subjects for which there is information, there were 46 disease progression events. 16 disease progress events were from the CD24Fc treatment arm, 30 were from the placebo arm. The cumulative progression rate estimated by the layered Cox-Model for day 29 was 16.2% in the CD24Fc arm, and 30.6% in the placebo arm. The hazard ratio was 0.488 (95% CI: 0.266 to 0.896) in FIG. 26. P value=0.021.

Among the 46 disease progression events, 26 subjects expired within 28 days. 5 patients were extubated and achieved clinical improvement and recovery within the 28 days from randomization. The remaining 15 patients were alive as the study completion D29 visits or as cut-off date of Aug. 27, 2020 if the D29 visit day was not reached.

All-Cause Mortality

All-cause mortality at Day 15 and Day 29 were calculated and reported. The Mantel-Haenszel stratum-weighted estimator of the risk difference were used to compare the treatments. Time to mortalities were listed by subject.

The probability function of mortalities were estimated by the Kaplan-Meier method. The median time and its 95% confidence interval for each group were reported. The 95% confidence interval for the median time were estimated using the Brookmeyer-Crowley method with log-log function conversion to achieve a normal approximation. The mortality rates were estimated by the layered Cox-Model. Cumulative mortality rates estimated by the KM method for day 29 and the 95% confidence intervals were reported.

Among the 197 subjects for which there is information, 26 patients expired within 28 days as of the cut-off date. Before randomization, 21 (81%) had baseline MAID score of 3; 5 (19%) had baseline NIAID score of 4.

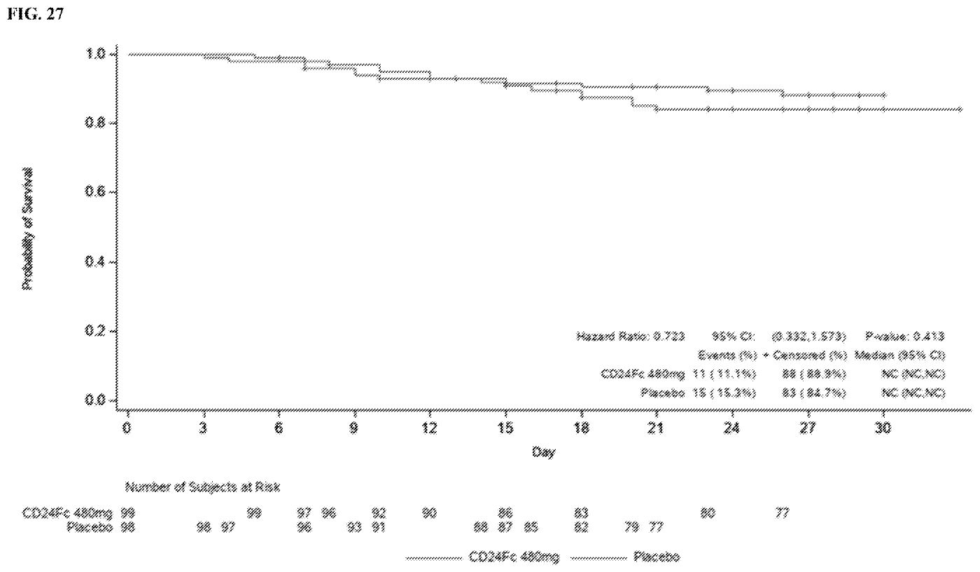

There were 15 deaths from the placebo arm and 11 deaths from the CD24Fc arm. The mortality rate estimated by K-M method at Day 15 was 8.16% in the placebo arm, 8.08% in the CD24Fc arm, and Day 29 is 15.3% in the placebo arm and 11.1% in the CD24Fc arm. The hazard ratio was 0.723 (95% CI: 0.332 to 1.573) in FIG. 27. P value=0.413.

There were 4 additional deaths after Day 29 from the randomization that were censored at Day 29 in the above analysis, 3 from the placebo arm and 1 from the CD24Fc arm.

Additional two deaths were reported as of 9/17/2020, both in the placebo arm.

In total, 20 deaths were reported in the placebo arm, and 12 deaths were reported in the CD24Fc arm OR=0.54 (95% CI 0.24-1.16).

The mortality cases as of the cut-off date were described in the mortality narratives in the following section.

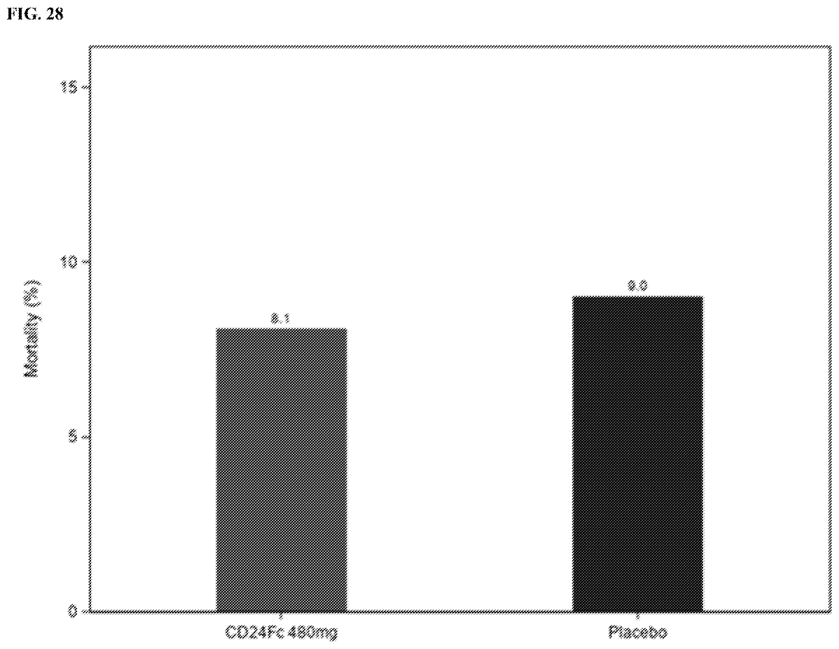

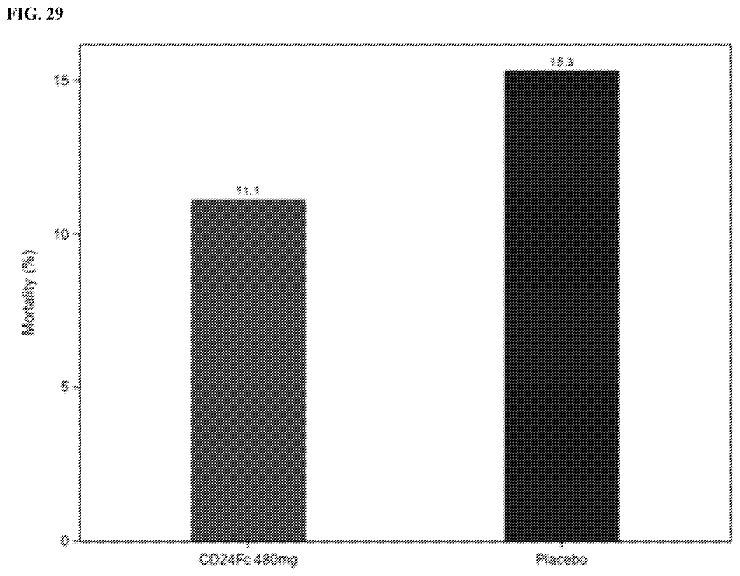

FIGS. 28 and 29 illustrate the mortality rates at D15 and D29 by treatment assignment.

Safety Analysis

Severe Adverse Events

All Grade 3 to 5 adverse events were coded by the most current version of MedDRA thesaurus. Treatment-emergent adverse events (TEAE) are defined as adverse events with a start date on or after the dose of the study drug and no later than the earliest of the following: 1) the date for the end of study day (Day 29); 2) the date of the early discontinuation to the study; 3) date for the DSMB data-cut. An adverse event or suspected adverse reaction is considered a serious adverse event (SAE) if, in the view of either the investigator or sponsor in this COVID-19 clinical trial, it results in any of the following outcomes: death, or a life-threatening adverse event that requires admission to the ICU, or organ failures.

The number of SAE and ADR cases, incidence and number of cases were summarized for each arm. SAEs and serious TEAEs were summarized by 1) system organ classification (SOC), preferred term (PT) and treatment arm, and 2) SOC, PT, CTCAE severity and treatment arm. SAEs, leading to study discontinuation and deaths, were listed by subject.

Adverse events severity were graded per CTCAE, as follows:

TABLE 10

| CTCAE Grade | Description |
|---|---|
| Grade 1 | Mild; asymptomatic or mild symptoms; clinical or diagnostic |
| | observations only; intervention not indicated |
| Grade 2 | Moderate; minimal, local or noninvasive intervention indicated; |
| | limiting age appropriate instrumental activities of daily living (ADL; |
| | i.e., preparing meals, shopping for groceries or clothes, using the |
| | telephone, managing money, etc) |
| Grade 3 | Severe or medically significant but not immediately life-threatening; |
| | hospitalization or prolongation of hospitalization indicated; disabling; |
| | limiting self-care ADL (i.e., bathing, dressing and undressing, feeding |
| | self, using the toilet, taking medications, and not bedridden) |
| Grade 4 | Life-threatening consequences; urgent intervention indicated |
| Grade 5 | Death related to AE |

TABLE 11
Fatal Serious Adverse Events

| | Arm A | Arm B | | | |
|---|---|---|---|---|---|
| | CD24Fc 480 mg | Placebo | | Overall | |
| System Organ Class | (N = 97) | (N = 97) | | (N = 194) | |
| Preferred Term | n (%) | n (%) | | n (%) | |
| Number of Subjects with Any Fatal SAE | 9 (9.3) | 13 | (13.4) | 22 | (11.3) |
| Number of Fatal SAEs | 10 | 13 | | 23 | |
| Cardiac disorders | 2 (2.1) | 3 | (3.1) | 5 | (2.6) |
| Cardiac arrest | 1 (1.0) | 1 | (1.0) | 2 | (1.0) |
| Cardio-respiratory arrest | 1 (1.0) | 2 | (2.1) | 3 | (1.5) |
| General disorders and administration site | 3 (3.1) | 3 | (3.1) | 6 | (3.1) |
| conditions | | | | | |
| Death | 2 (2.1) | 3 | (3.1) | 5 | (2.6) |
| Multiple organ dysfunction syndrome | 1 (1.0) | 0 | | 1 | (0.5) |
| Infections and infestations | 1 (1.0) | 0 | | 1 | (0.5) |
| COVID-19 | 1 (1.0) | 0 | | 1 | (0.5) |
| Renal and urinary disorders | 1 (1.0) | 0 | | 1 | (0.5) |
| Acute kidney injury | 1 (1.0) | 0 | | 1 | (0.5) |
| Respiratory, thoracic and mediastinal | 3 (3.1) | 7 | (7.2) | 10 | (5.2) |
| disorders | | | | | |
| Hypoxia | 1 (1.0) | 1 | (1.0) | 2 | (1.0) |
| Respiratory failure | 2 (2.1) | 6 | (6.2) | 8 | (4.1) |

TABLE 12
Non-Fatal Serious Adverse Events

| | Arm A | | Arm B | | |
|---|---|---|---|---|---|
| | CD24Fc 480 mg | | Placebo | Overall | |
| System Organ Class | (N = 97) | | (N = 97) | (N = 194) | |
| Preferred Term | n (%) | | n (%) | n (%) | |
| Number of Subjects with Any Non-Fatal | 11 | (11.3) | 7 (7.2) | 18 | (9.3) |
| SAE | | | | | |
| Number of Non-Fatal SAEs | 13 | | 7 | 20 | |
| Blood and lymphatic system disorders | 4 | (4.1) | 0 | 4 | (2.1) |
| Anaemia | 4 | (4.1) | 0 | 4 | (2.1) |
| Gastrointestinal disorders | 1 | (1.0) | 0 | 1 | (0.5) |
| Gastrointestinal haemorrhage | 1 | (1.0) | 0 | 1 | (0.5) |
| Infections and infestations | 0 | | 1 (1.0) | 1 | (0.5) |
| Escherichia bacteraemia | 0 | | 1 (1.0) | 1 | (0.5) |
| Investigations | 1 | (1.0) | 0 | 1 | (0.5) |
| Pulse absent | 1 | (1.0) | 0 | 1 | (0.5) |
| Neoplasms benign, malignant and | 1 | (1.0) | 0 | 1 | (0.5) |
| unspecified (incl cysts and polyps) | | | | | |
| Chronic lymphocytic leukaemia | 1 | (1.0) | 0 | 1 | (0.5) |
| Nervous system disorders | 1 | (1.0) | 1 (1.0) | 2 | (1.0) |
| Cerebrovascular accident | 0 | | 1 (1.0) | 1 | (0.5) |
| Hypoaesthesia | 1 | (1.0) | 0 | 1 | (0.5) |
| Renal and urinary disorders | 1 | (1.0) | 1 (1.0) | 2 | (1.0) |
| Acute kidney injury | 1 | (1.0) | 1 (1.0) | 2 | (1.0) |
| Respiratory, thoracic and mediastinal | 3 | (3.1) | 4 (4.1) | 7 | (3.6) |
| disorders | | | | | |
| Dyspnoea | 1 | (1.0) | 0 | 1 | (0.5) |
| Hypoxia | 0 | | 1 (1.0) | 1 | (0.5) |
| Laryngeal oedema | 1 | (1.0) | 0 | 1 | (0.5) |
| Pneumothorax | 1 | (1.0) | 0 | 1 | (0.5) |
| Respiratory distress | 0 | | 2 (2.1) | 2 | (1.0) |
| Respiratory failure | 0 | | 1 (1.0) | 1 | (0.5) |
| Vascular disorders | 1 | (1.0) | 0 | 1 | (0.5) |
| Peripheral ischaemia | 1 | (1.0) | 0 | 1 | (0.5) |

1. A method of treating or preventing a viral pneumonia in a subject in need thereof, comprising administering a CD24 protein to the subject.

2. The method of claim 1, wherein the pneumonia is caused by an influenza virus, a parainfluenza virus, a respiratory syncytial virus, or a human immunodeficiency virus.

3. The method of claim 1 or 2, wherein the pneumonia is caused by a secondary bacterial infection following a viral infection.

4. The method of claim 1, wherein the CD24 protein comprises a mature human CD24 polypeptide or a variant thereof comprising a sequence set forth in SEQ ID NO: 1 or 2.

5. (canceled)

6. The method of claim 4, wherein the CD24 protein comprises a protein tag, wherein the protein tag is fused at the N-terminus or C-terminus of the CD24 protein and comprises a portion of a mammalian immunoglobulin (Ig) protein.

7. (canceled)

8. The method of claim 6, wherein the portion of the mammalian Ig protein is a Fc region, wherein the Ig protein is human comprising a hinge region and CH2 and CH3 domains of a human Ig protein selected from the group consisting of IgG1, IgG2, IgG3, IgG4, and IgA, and the Fc region comprises a hinge region and CH2, CH3 and CH4 domains of IgM.

9. (canceled)

10. (canceled)

11. (canceled)

12. The method of claim 1, wherein the amino acid sequence of the CD24 protein comprises a sequence set forth in SEQ ID NO: 6, 11, or 12.

13. The method of claim 0, wherein the amino acid sequence of the CD24 protein consists of the sequence set forth in SEQ ID NO: 6, 11, or 12.

14. The method of claim 1, wherein the CD24 protein is produced using a eukaryotic protein expression system comprising a vector contained in a Chinese Hamster Ovary cell line or a replication-defective retroviral vector.

15. (canceled)

16. (canceled)

17. The method of claim 1, wherein the CD24 protein is soluble and/or glycosylated.

18. (canceled)

19. Use of a CD24 protein in the manufacture of a medicament for treating or preventing a viral pneumonia in a subject.

20. The use of claim 19, wherein the pneumonia is caused by an influenza virus, a parainfluenza virus, a respiratory syncytial virus, a human immunodeficiency virus, or the pneumonia is caused by a secondary bacterial infection following a viral infection.

21. (canceled)

22. The use of claim 19, wherein the CD24 protein comprises a mature human CD24 polypeptide or a variant thereof.

23. The use of claim 22, wherein the mature human CD24 polypeptide or variant thereof comprises a sequence set forth in SEQ ID NO: 1 or 2.

24. (canceled)

25. (canceled)

26. The use of claim 23, wherein the CD24 protein comprises a protein tag, wherein the protein tag is fused at the N-terminus or C-terminus of the CD24 protein.

27. The use of claim 26, wherein the protein tag comprises a portion of a mammalian immunoglobulin (Ig) protein and the portion of the mammalian Ig protein is a Fc region, wherein the Ig protein is human.

28. (canceled)

29. (canceled)

30. The use of claim 27 wherein the Fc region comprises a hinge region and CH2 and CH3 domains of a human Ig protein selected from the group consisting of IgG1, IgG2, IgG3, IgG4, and IgA.

31. The use of claim 27, wherein the Fc region comprises a hinge region and CH2, CH3 and CH4 domains of IgM.

32. The use of claim 19, wherein the amino acid sequence of the CD24 protein comprises a sequence set forth in SEQ ID NO: 6, 11, or 12.

33. The use of claim 22, wherein the amino acid sequence of the CD24 protein comprises a sequence set forth in SEQ ID NO: 6, 11, or 12.

34. (canceled)

35. (canceled)

36. (canceled)

37. The use of claim 19, wherein the CD24 protein is produced using a eukaryotic protein expression system.

38. The use of claim 37, wherein the expression system comprises a vector contained in a Chinese Hamster Ovary cell line or a replication-defective retroviral vector.

39. The use of claim 38, wherein the replication-defective retroviral vector is stably integrated into the genome of a eukaryotic cell.

40. The use of claim 19, wherein the CD24 protein is soluble and/or glycosylated.

41. (canceled)

42-63. (canceled)


SEQUENCE LISTING (12 sequences)

SEQ ID NO: 1 - protein, 31 aa, Homo sapiens
  VARIANT (31)...(31): Valine or Alanine
SETTTGTSSNSSQSTSNSGLAPNPTNATTKX

SEQ ID NO: 2 - protein, 30 aa, Homo sapiens
SETTTGTSSNSSQSTSNSGLAPNPTNATTK

SEQ ID NO: 3 - protein, 27 aa, Mus musculus
NQTSVAPFPGNQNISASPNPTNATTRG

SEQ ID NO: 4 - protein, 26 aa, Homo sapiens
MGRAMVARLGLGLLLLALLLPTQIYS

SEQ ID NO: 5 - protein, 287 aa, Artificial
  Fusion protein
MGRAMVARLGLGLLLLALLLPTQIYSSETTTGTSSNSSQSTSNSGLAPNPTNATTKPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 261 aa, Artificial
  Fusion protein
SETTTGTSSNSSQSTSNSGLAPNPTNATTKPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 231 aa, Homo sapiens
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 288 aa, Artificial
  Fusion protein
MGRAMVARLGLGLLLLALLLPTQIYSSETTTGTSSNSSQSTSNSGLAPNPTNATTKVPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 288 aa, Artificial
  Fusion protein
MGRAMVARLGLGLLLLALLLPTQIYSSETTTGTSSNSSQSTSNSGLAPNPTNATTKAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 29 aa, Macaca fascicularis
TVTTSAPLSSNSPQNTSTTPNPANTTTKA

SEQ ID NO: 11 - protein, 262 aa, Artificial
  Fusion protein
SETTTGTSSNSSQSTSNSGLAPNPTNATTKVPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 262 aa, Artificial
  Fusion protein
SETTTGTSSNSSQSTSNSGLAPNPTNATTKAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK